(19) United States
(12) Patent Application Publication
(10) Pub. No.: US 2018/0244901 A1
(43) Pub. Date: Aug. 30, 2018

(54) THERMOFORMED POLYMERIC ARTICLES CONTAINING AN ADDITIVE

(71) Applicant: DIANIA TECHNOLOGIES LIMITED, Galway (IE)

(72) Inventors: Sinéad KENNY, Galway (IE); Michael MALKOCH, Täby (SE)

(21) Appl. No.: 15/531,907

(22) PCT Filed: Dec. 22, 2016
(86) PCT No.: PCT/EP2016/082449

§ 371 Date: May 31, 2017

(30) Foreign Application Priority Data
Dec. 23, 2015 (IE) 2015/04538

(51) Int. Cl. C08L 23/06; C08L 87/00; A61L 29/12; C08G 63/06; B29C 47/00; B29C 71/02; B29C 43/00

(52) U.S. Cl. CPC C08L 23/06; C08L 87/005; A61L 29/126; C08G 63/06; B29C 47/0004; B29K 2023/065; B29C 43/003; C08L 2205/03; C08L 2207/062; B29C 47/0023; B29C 71/02

(57) ABSTRACT

The properties of a thermoformed polymeric article are modified by incorporation of an additive in a thermoplastic/thermoplastic elastic host matrix. The additive comprises a polydispersed hyperbranched polymer (HBP) or a branched monodispersed dendritic polymer (DP). The HBP or DP is linked to a plurality of oligomer chains. The additive migrates to the surface of the article during the thermoforming process.

### FIELD OF THE INVENTION

The invention relates to thermoformed articles with enhanced properties, especially for use in medical applications.

### BACKGROUND OF THE INVENTION

Tubing used in medical and healthcare applications often includes components to adjust the properties of the tubing. In some cases the tubing includes a reinforcement such as fibers or wires, for example, braided high tensile steel wires to enhance torque performance, liners to reduce frictional forces and fillers to confer colour or radiopacity under fluoroscopy.

However the incorporation of extra parts brings additional risks associated with patient safety. For example, an issue often reported upon clinical follow-up of various devices is delamination associated with the use of polymer liners. Detachment either of the liner material or another device component inside a patients' anatomy can result in serious injury for the patient or even death, as per reported by the US Food and Drug Administration. An important contributing factor to this issue is the nature of attachment between the extra part to the base thermoformed component.

Hydrophilic coatings are typically applied to the outer surface of many devices to improve various properties at the surface. Generally, these coatings are covalently bonded to the surface of the thermoformed component via a primer layer. For example, U.S. Pat. No. 6,278,018 describes a reagent comprising a non-polymeric core molecule comprising an aromatic group, a first photoreactive species and at least one charged group which was employed to modify the properties of the surface in terms of lubricity, hemocompatibility, wettability/hydrophilicity. However, reported failures of these types of coatings include the generation of particulates during storage and delamination during use, with significant potential safety risks for the patient.

The addition of extra parts to the thermoformed component also either increases the profile or reduces the inner lumen size of the final medical device. A low profile and large inner diameter is a critical characteristic in the success of a device such as a catheter in which a reduced profile enables penetration of smaller vessels.

### SUMMARY OF THE INVENTION

According to the invention there is provided a method for producing a thermoformed article with enhanced properties, comprising the steps of:—
introducing an additive into a host polymer (or host polymer matrix) to form a polymer/additive composition; and
thermoforming the polymer/additive composition into the article.

The additive may comprise:—
a polydispersed hyperbranched polymer (HBP) linked to a plurality of oligomer chains; or
a branched monodispersed dendritic polymer (DP) linked to a plurality of oligomer chains.

The polydispersed hyperbranched polymer may have at least two reactive groups.

The branched monodispersed dendritic polymer may have at least two reactive groups.

The additive may have greater than 30 carbon atoms.

In one embodiment the thermoforming is effected by extrusion forming. The extrusion forming may comprise a single or twin screw.

In one embodiment the method comprises forcing the molten mixture through a die.

In one case the method comprises the step of heat treating the thermoformed article. The heat treatment may be carried out at a temperature between room temperature and the glass transition temperature (Tg) of the host polymer.

In one embodiment the method comprises the step of blending the additive with the host polymer prior to thermoforming.

The blending may in some cases be selected from the group comprising:—
mixing;
melt blending, including extrusion compounding; and
solution blending comprising mixing said host polymer with said additive in a mutual solvent followed by dispersion blending.

In one case the polydispersed hyperbranched polymer or the monodispersed dendritic polymer have short cores with two or more reaction groups and six or more reactive peripheral groups linked to many short oligomers, O_{A}, where A represents a monomer and O_{A} represents an oligomer comprising two or more monomers A.

In another case the polydispersed hyperbranched polymer or the monodispersed dendritic polymer have short cores with two or more reactive groups and six or more reactive peripheral groups linked to a number of short oligomers, O_{A}, O_{C}, where A and C each represent a monomer (A being a different monomer than C) and O_{A}, O_{C} represent the respective oligomers, present in a ratio O_{A}:O_{C} of from 1:100 to 100:1.

In one embodiment the additive comprises a core linear chain.

In one case the additive comprises a core linear chain comprising monomer A having one reactive group which is monosubstituted to either the polydispersed hyperbranched polymer or the monodispersed dendritic polymer which is linked to many short oligomer A chains (O_{A}).

In one case the additive comprises a core linear chain comprising monomer A having two reactive groups and being di-substituted to two branched polymer components comprising the polydispersed hyperbranched polymer or the monodispersed dendritic polymer which are linked to many short, oligomer A chains (O_{A}).

In another case the additive comprises a core linear chain comprising monomer A having four reactive groups and being tetra-substituted to four branched polymer components comprising either the polydispersed hyperbranched polymer or the monodisperse dendritic polymer with 2 or more reactive groups which are linked to many short, oligomer A chains (O_{A}).

In a further case the additive comprises a core linear chain comprising monomer A having greater than six reactive groups and being substituted to a plurality of branched polymer components comprising either the polydispersed hyperbranched polymer or the monodispersed dendritic polymer with 2 or more reactive groups which is linked to many short, oligomer A chains (O_{A}).

In one case the additive comprises a core linear chain comprising monomer A having one reactive group, which is monosubstituted to a branched polymer component comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C} of from 1:100 to 100:1.

In one case the additive comprises a core linear chain comprising monomer A having two reactive groups and being di-substituted to two branched polymer components comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C}, of from 1:100 to 100:1.

In another case the additive comprises a core linear chain comprising monomer A having four reactive groups and being tetra-substituted to four branched polymer components comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C} of from 1:100 to 100:1.

In a further case the additive comprises a core linear chain comprising monomer A having a plurality of reactive groups and being substituted to a plurality of branched polymer components comprising either the polydispersed hyperbranched polymer or the monodisperse dendritic polymer with 2 or more reactive groups which is linked to a variety of many short oligomers, O_{A} and O_{C}, where A and C represent each monomer (A being a different monomer than C) and O_{A}, O_{C} represent the respective oligomers, present in a ratio O_{A}:O_{C} of from 1:100 to 100:1.

In some cases the additive, e.g., the linear chain of the additive, comprises at least one reactive group.

The oligomer(s) of the additive may, for example, comprise fluorinated, siliconized, alkyl and/or aliphatic units.

The linear chains or/and the oligomers may be fluorinated chains (such as vinylidene fluoride (VDF) including hexafluoropropylene, tetrafluoroethylene (TFE) and their copolymers including perfluoroalkyl vinyl esters such as perfluorooctanoic acid), that are thermoplastic in nature.

In one case the linear chains or/and the oligomers are siliconized chains including polymeric organosilicon compounds such as poly(dimethyl siloxane).

In another case the linear chains or/and the oligomers comprise alkyl, alkene, and/or alkyne chains, such as triglycerides or unsaturated fatty acids.

In some cases the linear chain or/and the oligomers are selected from acetyl, acetylene, adipic acid, acrylamide (acrylic amide), polyvinylpyrrolidone, poly(ethylene glycol)s, poly(propylene glycol)s, poly(ethylene glycol) monoalkyl ethers, and poly(propylene glycol) monoalkyl ethers.

In some instances the host polymer (which also may be referred to herein as a matrix polymer) is a polymer selected from one or more of the group comprising polyolefins, polystyrenes, polyesters, polyamides polyethers, polysulfones, polycarbonates, polyureas, polyurethanes, polysiloxanes and thermoplastic polymers including blends of thermoplastic polymers with other thermoplastics or copolymers or blends thereof and thermoplastic elastomers including blends of thermoplastic polymers with other thermoplastics or copolymers or blends thereof.

The additive may migrate to the surface of the resultant thermoformed article during the thermoforming process, causing a change to the surface properties of the resultant article compared to the surface properties of a thermoformed article formed from the host polymer alone.

There may be spontaneous migration/segregation due to an enthalphic mechanism—especially if the host polymer and the additive have incompatible/immiscible elements (e.g., the host polymer having chemical and/or physical properties that are incompatible/immiscible with the host polymer).

Subsequent to the thermoforming process, further exposure to temperatures above ambient temperatures during an annealing step, may result in additional migration of the additive to the surface of the resultant article, causing a change to the surface properties of the resultant article compared to the surface properties of the article immediately after the thermoforming process.

The additive may act as a transport system within the matrix polymer, transporting or migrating the specific terminal groups of the additive to the surface of the final formed article.

The additive may comprise oligomer groups which confer specific properties to the surface of the host polymer, including changes in the surface energy and/or surface tension.

In one case the additive comprises agents having antimicrobial properties and/or configured to impart antimicrobial effects to the host polymer or thermoformed article, such as zinc oxide compounds, silver compounds, nanosilver, silver sulfadiazine, silver nitrate, silver oxide, sulphonamides, amines and their salts, beta-lactams (pencillins and cephalosporins), Ex. penicillin G, cephalothin) and benzimidazole derivatives, semisynthetic penicillin (Ex. ampicillin, amoxycillin), clavulanic acid (Ex. clavamox is clavulanic acid plus amoxycillin), monobactams (Ex. aztreonam), carboxypenems (Ex. imipenem), aminoglycosides (Ex. streptomycin), gentamicin, glycopeptides (Ex. vancomycin), lincomycins (Ex. clindamycin), macrolides (Ex. erythromycin), polypeptides (Ex. polymyxin), bacitracin, polyenes (Ex. amphotericin), nystatin, rifamycins (Ex. rifampicin), tetracyclines (Ex. tetracycline), semisynthetic tetracycline (Ex. doxycycline), chloramphenicol (Ex. chloramphenicol), pyrazinamide, and sulfa drugs (ex. sulfonamide), antiseptic agents such as chlorhexidine, iodine/iodophors, and triclosan. Still further non-limiting examples include quaternary ammonium compounds, phosphate imidazolinium compounds, dimethyl benzyl ammonium chloride compounds, dimethyl ethylbenzyl ammonium chloride, alkyl dimethyl ammonium chloride, paradiisobutylphenoxyethoxyethyl dimethyl benzyl ammonium chloride, poly (hexamethylene biguanide hydrochloride), and tetramine compounds. Further non-limiting examples include essential oils such as oregano oil, tea tree oil (melaleuca Oil), mint oil, sandalwood oil, clove oil, nigella sativa (black cumin) oil, onion oil (allium cepe)—phytoncides, leleshwa oil, lavender oil, lemon oil, eucalyptus oil, peppermint oil, and cinnamon oil. Further non-limiting examples include nitrofuranes such as nitrofurantoin and nitrofurazone.

In some cases the additive may further comprise agents having anti-thrombogenic properties and/or configured to impart anti-thrombogenic properties to the host polymer or thermoformed article, such as anticoagulant and/or anti-platelet agents, for example non-limiting examples of heparin group (platelet aggregation inhibitors), methacryloyloxyethyl phosphorylcholine polymer, polyphloretinphosphate, heparin, heparan sulphate, hirudin, lepirudin, dabigatran, bivalirudin, fondaparinux, ximelagatran, direct thrombin inhibitors, argatroban, melagatran, ximelagatran, desirudin, defibrotide, dermatan sulfate, fondaparinux, rivaroxaban, antithrombin III, bemiparin, dalteparin, danaparoid, enoxaparin, nadroparin, parnaparin, reviparin, sulodexide, tinzaparin, vitamin K antagonists, acenocoumarol, clorindione, dicumarol (dicoumarol), diphenadione, ethyl biscoumacetate, phenprocoumon, phenindione, tioclomarol, warfarin, platelet aggregation inhibitors, abciximab, acetylsalicylic acid (aspirin), aloxiprin, beraprost, ditazole, carbasalate calcium, cloricromen, clopidogrel, dipyridamole, eptifibatide, indobufen, iloprost, picotamide, prasugrel, prostacyclin, ticlopidine, tirofiban, treprostinil, triflusal, enzymes, alteplase, ancrod, anistreplase, brinase, drotrecogin alfa, fibrinolysin, protein C, reteplase, saruplase, streptokinase, tenecteplase, urokinase, chelators, citrate, EDTA, and oxalate.

In some cases the additive may further comprise agents having anti-inflammatory properties and/or configured to impart anti-inflammatory properties to the host polymer or thermoformed article, such as non-steroidal anti-inflammatory drugs, salicylates (such as aspirin (acetylsalicylic acid), diflunisal, ethenzamide), arylalkanoic acids (such as diclofenac, indometacin, sulindac), 2-arylpropionic acids (profens) (such as carprofen, flurbiprofen, ibuprofen, ketoprofen, ketorolac, loxoprofen, naproxen, tiaprofenic acid), N-arylanthranilic acids (fenamic acids) (such as mefenamic acid), pyrazolidine derivatives (such as phenylbutazone), oxicams (such as meloxicam, piroxicam), coxibs (such as celecoxib, etoricoxib, parecoxib, rofecoxib, valdecoxib), sulphonanilides (such as nimesulide), diclofenac, flurbiprofen, ibuprofen, indometacin, ketoprofen, naproxen, piroxicam, and eicosanoids. Further non-limiting examples include any of a group of substances that are derived from arachidonic acid, including leukotrienes, thromboxanes, and prostaglandins. Further non-limiting examples include immunosuppressive drugs. Further non-limiting examples include analogues of rapamycin, such as tacrolimus (FK-506), sirolimus and everolimus, paclitaxel, docetaxel, and erlotinib.

In one case the additive, the host polymer, or the polymer/additive composition further comprises a radiopaque filler, a pigment, and/or a dye.

The polymer, the additive, and the polymer/additive composition each may be in the form of oil, waxy solids, powders, pellets, granules or any other thermoformable form.

The method of the invention may comprise the step of blending the additive with the host polymer prior to thermoforming. Blending may, for example, be selected from the group consisting of mixing, melt blending, including extrusion compounding, solution blending, and/or mixing said host polymer with said additive in a mutual solvent followed by dispersion blending.

The thermoforming method used in the invention may be any suitable thermoforming method that results in an article with surfaces enriched in the additive. The thermoforming may, in particular, be effected by extrusion forming, including multilayer extrusion forming, profile extrusion forming and the like means, utilising either a twin or single screw and a die through which the molten polymer is forced to form a continuous profile. The profile may be any shape including solid (such as a planar sheet or cylinder) or hollow (such as a tube which may have straight or curved edges). Press forming and vacuum press forming may also be utilised to produce specially formed products, whereby the polymer in a solid form is formed under pressure. Crystalline based polymers may be formed at temperatures approximately in the region of 10 to 40° C. above the melting point of the crystalline polymer. Amorphous materials may be formed at approximate temperatures in the region of 80 to 150° C. above the glass transition temperature of the amorphous polymer.

Downstream and upstream equipment utilised in the extrusion compounding and forming processes can include drying systems, gravimetric dosing and feeding systems, vacuum calibration/cooling water bath, haul-off systems and in-line measurement systems.

In one case the method comprises forcing the admixture through a die to increase the shear force at work on the composition during the thermoforming. process.

Also provided is an article which is thermoformed by a method of the invention. The article may be hollow or solid and may have straight or curved edges. In one case the article is a medical device. In some cases the article is tubular such as a catheter or sheath.

The invention also provides an additive for a thermoformable polymer matrix which comprises:—
a polydispersed hyperbranched polymer (HBP) linked to a plurality of oligomer chains; or
a branched monodispersed. dendritic polymer (DP) linked to a plurality of oligomer chains;
wherein the polydispersed hyperbranched polymer or the branched monodispensed dendritic polymer has at least two reactive groups.

The invention also provides a polydispersed hyperbranched polymer (HBP) having at least two reactive groups, the polydispersed hyperbranched polymer being linked to a plurality of oligomer chains; or a branched monodispersed dendritic polymer (DP) having at least two reactive groups, the branched monodispersed dendritic polymer being linked to a plurality of oligomer chains.

In the branched polymer (the polydispersed. hyperbranched polymer or the branched monodispersed dendritic polymer) or the additive comprising such branched polymer, one or more of the following may apply:—
the polydispersed hyperbranched polymer or the monodispersed dendritic polymer have short cores with two or more reaction groups and six or more reactive peripheral groups linked to many short oligomers, O_{A}, where A represents a monomer,
the polydispersed hyperbranched polymer or the monodispersed dendritic polymer have short cores with two or more reactive groups and six or more reactive peripheral groups linked to a number of short oligomers, O_{A}, O_{C}, where A and C each represent a monomer (A being a different monomer than C) and O_{A}, O_{C} represent the respective oligomers, present in a ratio O_{A}:O_{C} of from 1:100 to 100:1,
the polymer comprising a core linear chain,
the polymer comprising a core linear chain comprising monomer A having one reactive group which is monosubstituted to either the polydispersed hyperbranched polymer or the monodispersed dendritic polymer which is linked to many short oligomer A chains (O_{A}),
the polymer comprising a core linear chain comprising monomer A having two reactive groups and being di-substituted to two branched polymer components comprising the polydispersed hyperbranched polymer or the monodispersed dendritic polymer which are linked to many short, oligomer A chains (O_{A}),
the polymer comprising a core linear chain comprising monomer A and having four reactive groups, which is tetra-substituted to four branched polymer components comprising either the polydisperse hyperbranched polymer or the monodisperse dendritic polymer with 2 or more reactive groups which are linked to many short, oligomer A chains (O_{A}),
the polymer comprising a core linear chain comprising monomer A having greater than six reactive groups and being substituted to a plurality of branched polymer components comprising either the polydispersed hyperbranched polymer or the monodispersed dendritic polymer with 2 or more reactive groups which is linked to many short, oligomer A chains (O_{A}),
the polymer comprising a core linear chain comprising monomer A having one reactive group, which is monosubstituted to a branched polymer component comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C} of from 1:100 to 100:1,
the polymer comprising a core linear chain comprising monomer A having two reactive groups and being di-substituted to two branched polymer components comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C} of from 1:100 to 100:1,
the polymer comprising a core linear chain comprising monomer A having four reactive groups and being tetra-substituted to four branched polymer components comprising either the polydispersed hyperbranched polymer or the monodispersed dendritic polymer with 2 or more reactive groups which is linked to a plurality of short oligomers, O_{A} and O_{C}, where A and C each represent a monomer (A being a different monomer than C) and O_{A}, O_{C} represent the respective oligomers, present in a ratio O_{A}:O_{C} of from 1:100 to 100:1,
the polymer comprising a core linear chain comprising monomer A having a plurality of reactive groups and being substituted to a plurality of branched polymer components comprising, either the polydisperse hyperbranched polymer or the monodisperse dendritic polymer with 2 or more reactive groups which is linked to a variety of many short oligomers, O_{A} and O_{C}, where A and C represent each monomer (A being a different monomer than C) and O_{A}, O_{C} represent the respective oligomers, present in a ratio O_{A}:O_{C} of from 1:100 to 100:1.

In some cases the additive, e.g., the linear chain of the additive, comprises at least one reactive group.

The oligomer(s) of the additive may in some cases comprise fluorinated, siliconized, alkyl and/or aliphatic units.

In one case the linear chains or/and the oligomers are fluorinated chains (such as vinylidene fluoride (VDF) including hexafluoropropylene, tetrafluoroethylene (TFE) and their copolymers including perfluoroalkyl vinyl esters such as perfluorooctanoic acid), that are thermoplastic in nature.

The linear chains or/and the oligomers are in some cases siliconized chains including polymeric organosilicon compounds such as poly(dimethyl siloxane).

In some cases the linear chains or/and the oligomers comprise alkyl, alkene, and/or alkyne chains, such as triglycerides or unsaturated fatty acids.

In some examples the linear chain or/and the oligomers are selected from acetyl, acetylene, adipic acid, acrylamide (acrylic amide), polyvinylpyrrolidone, poly(ethylene glycol)s, poly(propylene glycol)s, poly(ethylene glycol) monoalkyl ethers, and poly(propylene glycol) monoalkyl ethers.

Also provided is a composition comprising an additive as described herein and a host polymer.

In some examples the host polymer (which also may be referred to herein as a matrix polymer) is a polymer selected from one or more of the group comprising polyolefins, polystyrenes, polyesters, polyamides polyethers, polysulfones, polycarbonates, polyureas, polyurethanes, polysiloxanes and thermoplastic polymers including blends of thermoplastic polymers with other thermoplastics or copolymers or blends thereof and thermoplastic elastomers including blends of thermoplastic polymers with other thermoplastics or copolymers or blends thereof.

The additive may migrate to the surface of the resultant thermoformed article during the thermoforming process, causing a change to the surface properties of the resultant article compared to the surface properties of a thermoformed article formed from the host polymer alone.

Subsequent to the thermoforming process, further exposure to temperatures above ambient, may result in additional migration of additive to the surface of the resultant article, causing a change to the surface properties of the resultant article compared to the surface properties of the article immediately after the thermoforming process.

The additive may act as a transport system within the matrix polymer, transporting or migrating the specific terminal groups of the additive (e.g., terminal groups of the polydispersed hyperbranched polymer or branched monodispersed dendritic polymer) to the surface of the final formed article.

The oligomer group(s) of the polydispersed hyperbranched polymer or branched monodispersed dendritic polymer may confer specific properties to the surface of the host polymer (e.g., the surface of the article formed from the host polymer), including changes in the surface energy and/or surface tension.

The composition may comprise agents having antimicrobial properties and/or configured to impart antimicrobial effects to the host polymer or thermoformed article, such as zinc oxide compounds, silver compounds, nanosilver, silver sulfadiazine, silver nitrate, silver oxide, sulphonamides, amines and their salts, beta-lactams (pencillins and cephalosporins) and benzimidazole derivatives, semisynthetic penicillin (Ex. ampicillin, amoxycillin), clavulanic acid (Ex. clavamox is clavulanic acid plus amoxycillin), monobactams (Ex. aztreonam), carboxypenems (Ex. imipenem), aminoglycosides (Ex. streptomycin), gentamicin, glycopeptides (Ex. vancomycin), lincomycins (Ex. clindamycin), macrolides (Ex. erythromycin), polypeptides (Ex. polymyxin), bacitracin, polyenes (Ex. amphotericin), nystatin, rifamycins (Ex. rifampicin), tetracyclines (Ex. tetracycline), semisynthetic tetracycline (Ex. doxycycline), chloramphenicol (Ex. chloramphenicol), pyrazinamide, and sulfa drugs (ex. sulfonamide), antiseptic agents such as chlorhexidine, iodine/iodophors, and triclosan. Still further non-limiting examples include quaternary ammonium compounds, phosphate imidazolinium compounds, dimethyl benzyl ammonium chloride compounds, dimethyl ethylbenzyl ammonium chloride, alkyl dimethyl ammonium chloride, paradiisobutylphenoxyethoxyethyl dimethyl benzyl ammonium chloride, poly (hexamethylene biguanide hydrochloride), and tetramine compounds. Further non-limiting examples include essential oils such as oregano oil, tea tree oil (melaleuca Oil), mint oil, sandalwood oil, clove oil, nigella sativa (black cumin) oil, onion oil (allium cepe)—phytoncides, leleshwa oil, lavender oil, lemon oil, eucalyptus oil, peppermint oil, and cinnamon oil. Further non-limiting examples include nitrofuranes such as nitrofurantoin and nitrofurazone.

The composition may comprise agents having anti-thrombogenic properties and/or configured to impart anti-thrombogenic properties to the host polymer or thermoformed article, such as anticoagulant and/or anti-platelet agents, for example non-limiting examples of heparin group (platelet aggregation inhibitors), methacryloyloxyethyl phosphorylcholine polymer, polyphloretinphosphate, heparin, heparan sulphate, hirudin, lepirudin, dabigatran, bivalirudin, fondaparinux, ximelagatran, direct thrombin inhibitors, argatroban, melagatran, ximelagatran, desirudin, defibrotide, dermatan sulfate, fondaparinux, rivaroxaban, antithrombin III, bemiparin, dalteparin, danaparoid, enoxaparin, nadroparin, parnaparin, reviparin, sulodexide, tinzaparin, vitamin K antagonists, acenocoumarol, clorindione, dicumarol (dicoumarol), diphenadione, ethyl biscoumacetate, phenprocoumon, phenindione, tioclomarol, warfarin, platelet aggregation inhibitors, abciximab, acetylsalicylic acid (aspirin), aloxiprin, beraprost, ditazole, carbasalate calcium, cloricromen, clopidogrel, dipyridamole, eptifibatide, indobufen, iloprost, picotamide, prasugrel, prostacyclin, ticlopidine, tirofiban, treprostinil, triflusal, enzymes, alteplase, ancrod, anistreplase, brinase, drotrecogin alfa, fibrinolysin, protein C, reteplase, saruplase, streptokinase, tenecteplase, urokinase, chelators, citrate, EDTA, and oxalate.

In some cases the additive may further comprise agents having anti-inflammatory properties and/or configured to impart anti-inflammatory properties to the host polymer or thermoformed article, such as non-steroidal anti-inflammatory drugs, salicylates (such as aspirin (acetylsalicylic acid), diflunisal, ethenzamide), arylalkanoic acids (such as diclofenac, indometacin, sulindac), 2-arylpropionic acids (profens) (such as carprofen, flurbiprofen, ibuprofen, ketoprofen, ketorolac, loxoprofen, naproxen, tiaprofenic acid), N-arylanthranilic acids (fenamic acids) (such as mefenamic acid), pyrazolidine derivatives (such as phenylbutazone), oxicams (such as meloxicam, piroxicam), coxibs (such as celecoxib, etoricoxib, parecoxib, rofecoxib, valdecoxib), sulphonanilides (such as nimesulide), diclofenac, flurbiprofen, ibuprofen, indometacin, ketoprofen, naproxen, piroxicam, and eicosanoids. Further non-limiting examples include any of a group of substances that are derived from arachidonic acid, including leukotrienes, thromboxanes, and prostaglandins. Further non-limiting examples include immunosuppressive drugs. Further non-limiting examples include analogues of rapamycin, such as tacrolimus (FK-506), sirolimus and everolimus, paclitaxel, docetaxel, and erlotinib.

The composition may comprise at least one additive, at least one host polymer, and optionally one or more active agents or bound agents as described herein.

The composition may comprise a radiopaque filler, a pigment, and/or a dye.

The composition may be in the form of powders, pellets, granules or any other thermoformable form.

Also described is a process comprising the step of thermoforming a composition as described.

The process may comprise the step of blending the composition prior to thermoforming. In some examples blending is selected from the group consisting of mixing, melt blending, solution blending, and/or mixing said host polymer with said additive in a mutual solvent and dispersion blending.

The process in some cases may include forcing the admixture through a die to increase the shear force at work on the composition during the thermoforming process.

The invention also provides a thermoformed polymeric article including a self-segregating branched polymer hybrid with many chain ends, whereby the branched polymer component is linked to long linear polymer chains and/or many small oligomer chains and has a concentration profile at the surface of the formed article.

In the invention, thermoformed articles may comprise a thermoplastic/thermoplastic elastic matrix and a minor amount (e.g., between 0.1 and 30%, between 0.1 and 15%, between 1.5 and 7%, or between 1.5 and 6% % by weight) of a branched-hybrid polymer additive. In the final thermoformed article the additive is distributed in the polymer matrix, concentrated at the surfaces with a reducing concentration gradient towards the bulk of the polymer matrix, resulting in modified surface properties with respect to the bulk.

The hyperbranched and dendritic polymers used in the invention may have the ability to segregate spontaneously to a polymer surface, modify surface properties such as rheological properties and reduce viscosity of the polymer melt during thermoforming processes.

The surface properties of branched polymers (such as polydispersed hyperbranched polymers and branched monodispersed dendritic polymers) depend on the functionality of terminal end-groups of the branched polymers, with said surface properties scaling with the number of terminal segments which are located at the periphery of these macromolecules. The number of terminal end groups is indicated by the molecular weight of the branched polymer, and by the degree of branching of the branched polymer element. Equipping the many chain ends (i.e. terminal points) of said branched polymers with targeted small linear chains which possess specific properties to form a branched hybrid polymer, and consequently blending such a branched hybrid polymer in a polymer matrix, is believed to surmount the energetic barrier and enthalpically segregate the branched polymer (e.g., a higher-energy species) to the surface of the final thermoformed article. Branched polymers are more compact, typically, depending on their generation/size, have little/no entanglements and therefore migrate much faster than a linear polymer. Linear polymers are more likely to get trapped in the host polymer matrix due to interactions between their chains and those of the host polymer resulting in entanglement, trapping the linear polymer in the bulk of the host polymer matrix. Therefore there are a number of factors which influence the migration of the additive to the surface, including:

Branched Polymer Element
Size of the branched polymer element: as the degree of branching of the branched element increases, the number of terminal end groups also increases. However this increase in the number of terminal end groups has to be balanced against the effect of increased molecular weight of the branched polymer element on the migration of the additive.
Polarity of the branched polymer element: differences between the polarity of the additive and the host matrix may influence migration of the additive to the surface of the host matrix. Lower energy polymers are expected to reside at the air/surface interface.
Differences in the polarity between the branched polymer element and the terminal end groups also is expected to play a role in the migration of the additive to the surface, e.g., by producing an additional enthalpic attraction of chains to the surface.
Architecture: the symmetry and size of the branched polymer element is known to influence the migration. The shape and its architecture of the additive may reduce the potential for entanglement or ensure that the branched polymer element does not get entangled with linear chains of the host polymer. The size of the branched polymer element as discussed may be influential during thermoforming processes, whereby the effect of shear on the branched polymer element influences its ability to migrate to areas of higher stress within the molten polymer.
Shape: surface area to volume ratio whereby more free volume can increase solubility and diffusivity of the additive.

Terminal End Group
Number of terminal end groups: A relatively small quantity of additive can provide a large modification of surface properties when functional terminal groups are delivered to the surface via the branched polymer element.
Polarity of terminal end groups: again as discussed chemical heterogeneity with the branched polymer element and the host polymer can enable enthalpic migration to the air/surface interface of the final product.
Degrees of freedom of terminal end groups: Due to the free volume enjoyed by the terminal end groups compared to the branched polymer element, the terminal end groups may confer substantial influence over the surface properties of the final product.

Host Matrix
Molecular weight differences with additive: when subjected to shear forces, for example during a thermoforming process, species with a higher M_{w} may experience a larger decrease in configurational entropy, (i.e.) the molecules will experience higher levels of compression/deformation compared to lower M_{w} components. Hence during the extrusion process higher M_{w} components tend to stay in regions of lower stress whilst lower M_{w} components tend to be attracted to areas of higher stress, towards the surfaces and walls of the extruder equipment, enriching, the surface to maintain lower surface energy.
Crystallinity: in a highly crystalline polymer, mobility of an additive would be expected to be less than in a less crystalline or amorphous polymer, as the movement of the additive would be interrupted by the crystalline portions of the polymer, with motion occurring more readily in amorphous regions.
Polarity Heterogeneity: as discussed above.

One aspect of this present invention provides a thermoformed article having reduced friction surfaces imparted via an additive whose concentration profile increases outwards from the central regions of the article to the air/surface interface. The reduction in friction may be due to (a) preferential migration of the branched hybrid of the additive to the air/polymer interface due to (b) enthalpic differences between the additive and the host polymer matrix and (c) entropy via shearing imparted during compounding and thermoforming, processes resulting in (d) terminal end groups of the small linear chains on the branched polymer component of the additive, which, depending on their polarity, may impose certain surface properties specific to the desired end application of the thermoformed article. This preferential migration may ensure the specific terminal chains reside at the air/polymer interface where they may impart desired surface properties to the thermoformed article.

Other aspects of the invention involve the modification of the surface energy of the thermoformed article and/or inclusion of one or more active agents, such as antimicrobial agents to provide antimicrobial properties, e.g., to prevent infection. These active agents may be associated with the branched polymer additive by means of covalent bonding, ionic bonding, binding though charged groups, binding through polar groups, binding through van der Waals forces, colloidal stabilization, formation of organic-inorganic nanoparticles, formation of organic-inorganic micro-particles and/or dispersion or loading into the polymer structure.

In an additional aspect, the branched polymer additive may be modified with functional groups that may provide enhanced binding or loading of the active agent(s), or modified with other functional groups that may impart antimicrobial properties, prevent microbial adhesion to a surface, and/or facilitate the migration of the additive to the surface of the host polymer material during thermoforming. Such functional groups may include, but are not limited to hydroxyl, amines and their salts, carboxylic acids and ethers such as polyethylene glycol (PEG). As such the branched polymer may be tailored to be associated with specific active agents. As a non-limiting example, dendritic polymers with multiple polar end groups are known by several methods to stabilise silver nanoparticles. Typically, stable silver particles can be formed when a solution containing, a cationic silver species is treated with a reducing, agent in the presence of a polyol. As such, silver nitrate may form nanoparticles with a branched polymer additive containing multiple hydroxyl groups, when reduced by an appropriate reagent, providing a material that may be extruded with the host polymer to impact anti-microbial properties to the thermoformed material.

Cationic silver species such as silver nitrate form a salt when stirred with a carboxylic acid in an appropriate solvent. In some aspects of the present disclosure, for example, a branched polymer with multiple carboxylic acid end groups may be stirred with a solution of silver nitrate and isolated with the branched polymer with multiple associated cationic silver moieties, yielding an additive that may be extruded with a host polymer to generate a thermoformed. material with anti-microbial properties.

In another example a branched polymer additive may be functionalised with cationic amine end groups before extrusion with the host polymer, yielding a thermoformed material with anti-fouling properties that may prevent the growth of biomaterial on the surface of the thermoformed article and/or formation of a biofilm.

Similarly a branched polymer additive may be functionalised with anti-thrombus agents (e.g., to prevent bleeding), and/or may include one or more pharmaceutical drugs (e.g., for treatment of inflammation), radiopaque fillers (e.g., to enable observation under fluoroscopy), and/or pantone fillers (e.g., for aesthetic purposes in a thermoformed article that can be sterilised and stored for the required shelf life).

Modified branched polymer additives incorporated in thermoplastic/thermoplastic elastic resins are shown herein to alter surface characteristics of thermoformed articles made from such additive/resin compositions.

The ability to alter the surface characteristics is believed to occur due to a relatively high local concentration profile of the branched polymer additive at the first few surface nanometers of the thermoformed article. Various different mechanisms are believed to enable preferential segregation of such an additive to such a surface. Enthalpically driven segregation of a component from the bulk to the surface/air interface of a polymer mixture is believed to occur in order to decrease interfacial energy and minimize the overall free energy of the system. Here the driving force for surface migration is believed to be largely thermodynamic, where the component with the lowest critical surface tension rises to the air-polymer interface, thereby lowering the interfacial free energy.

Further, the miscibility and mobility of the components is believed to influence the kinetic driving force toward the interface. Incompatibility between polymers (e.g., incompatibility between the branched polymer additive and the host polymer) is believed to enhance this type of segregation. While the surface energy difference dictates which component(s) of a mixture segregate to the surface, χ, the interaction parameter, has a large effect on the degree of segregation among such components. Due to the nature of dendrimers, the structure of the dendrimer backbone can differ from that of the branches and terminal end groups. The combined contribution of each of the elements or portions of the branched polymer additive can aid surface segregation whilst also facilitating miscibility/immiscibility between the additive and host polymer matrix.

Conversely, another mode of surface migration may occur due to an entropic driving force. The magnitude of a dendritic polymer presence at the surface (e.g., the concentration of the branched polymer additive at or proximate the surface) may increase with an increasing number of chain ends and decrease with increased segmental crowding near the branch point, so called chain-end (conformational) driven segregation. Migration may occur as a result of a thermodynamic balancing between the host polymer matrix, the branched elements of the branched polymer additive within the host polymer matrix, functionalised terminal groups of the branched polymer additive, and/or the external environment. Manipulating a linear chain of a branched polymer additive to increase its number of chain ends, while decreasing segmental crowding in the branched polymer additive is expected to optimise the migration of such additives in a host polymer matrix.

During blending and extrusion processes, dendritic polymers may form a lubricating layer between the surfaces of the extrusion equipment and the bulk polymer material. Melt mixing is useful for polymer blend preparation. The migration of the branched polymer additive to the surface/air interface may be positively influenced by the mechanical forces sustained during mixing in the molten state in extruders or batch mixers. In the course of these thermal forming processes, a dendritic additive may be concentrated at the exterior surfaces of the host polymer liquid, which are frozen (immobilised) in-situ on cooling.

The invention also provides a thermoformed article comprising a host polymer and an additive comprising:—
a polydispersed hyperbranched polymer (HBP) having at least two reactive groups, the polydispersed hyperbranched polymer being linked to a plurality of oligomer chains; or
a branched monodispersed dendritic polymer (DP) having at least two reactive groups, the branched monodispersed dendritic polymer being linked to a plurality or oligomer chains, and wherein the surface of the article is enriched in the additive.

In one case a surface property of the host polymer is modified compared to the host polymer without the additive. The surface property, for example, may be surface tension and/or surface energy.

In some cases the surface property is one or more of anti-microbial, anti-thrombogenic, anti-inflammatory or radiopacity.

The article may be a medical device. The article may be a tubular article such as a catheter.

Medical devices include such devices as employed in sheaths, stents, delivery systems, decontamination barriers (in the form of a clinical and/or sterile barrier), medical clothing, imaging devices, skin therapy etc., with such devices employed for various durations, including transient, short-term, long-term or continuous use basis. Such devices include those employed in various applications including diagnostic, therapeutic, minimally invasive, invasive, surgical, intravascular, intervascular, intradermal or by way of natural anatomical orifice insertion into the human body.

The thermoformed articles may comprise tubing for use in such medical applications whereby the tubing is incorporated in such medical devices.

Tubular medical devices include those employed in sheaths, catheters, stents, delivery systems, imaging devices, skin therapy etc., with such devices employed for various durations, including transient, short-term, long-term or continuous use basis. Such devices include those employed in various applications including diagnostic, therapeutic, minimally invasive, invasive, surgical, intravascular, intervascular, intradermal or by way of natural anatomical orifice insertion into the human body.

In some cases the additive and the host polymer are blended and thermoformed by extrusion into the form of a string which may be cut into pellets. The pellets comprising the polymer additive matrix may then be further processed by thermoforming into a desired profile, such as a sheet or a hollow article such as a tube.

In another case the additive and the host polymer are blended and directly thermoformed into a desired profile, for example by extrusion and passing through a die which produces the desired profile such as a sheet or hollow article such as a tube.

### BRIEF DESCRIPTION OF THE DRAWINGS

The invention will be more clearly understood from the following description of some embodiments thereof, given by way of example only, with reference to the accompanying drawings, in which:

FIG. 1(a) shows the various classes of dendritic polymers, categorised according to the dispersion in their frameworks (i.e.) mono or poly;

FIG. 1 (b) shows a hyperbranched structure having a branched core with customisable chain periphery;

FIG. 2 shows the resultant molecular structures of the branched hybrid formed when for (i and ii) a branched polymer component (B) is connected to multiple short oligomers (O_{A} or/and O_{C}) as well as structures (iii to vii) which also include long linear polymer component(s) (A), where the number of reactive groups is 1 or more, are reacted during chemical synthesis. Resultant structures include:
i. a generic (B)—O_{A} “Hyperstar” polymer comprising of monodisperse or polydisperse branched components with short cores with functionality of 2 or more and 6 or more reactive perpherial groups linked to small, short oligomer A chains (O_{A});
ii. a generic (B)—O_{A} O_{C} “Hyperstar” polymer comprising of monodisperse or polydisperse branched components with short cores with functionality of 2 or more and 6 or more reactive perpherial groups are linked to small, short oligomer A chains (O_{A}) and small, short oligomer C chains (O_{C}); where the ratio of O_{A} oligomers, x, to O_{C} oligomers, y, is between 1-99%;
iii. a generic (AB)—O_{A} block copolymer comprising of a longer, linear chain (A) which is monosubstituted to a branched polymer component of either monodisperse or polydisperse structure with 2 or more reactive groups, which in turn is linked to small, short oligomer A chains (O_{A});
iv. a generic (BAB)—O_{A} block copolymer comprising of a longer, linear chain (A), which is di-substituted to two branched polymer components of either a monodisperse or polydisperse structure with 2 or more reactive groups, which in turn are linked to small, short oligomer A chains (O_{A});
v. a generic (AB₄)—O_{A} block copolymer comprising of a longer, linear chain (A), which is tetra-substituted to four branched polymer components of either monodisperse or polydisperse structure with 2 or more reactive groups which in turn are linked to small, short oligomer A chains (O_{A});
vi. a generic (AB)—(O_{A}), (O_{C})_{y} block copolymer comprising of a longer, linear chain (A), which is monosubstituted to a branched polymer component of either monodisperse or polydisperse structure with 2 or more reactive groups which in turn is linked to small, short oligomer A chains (O_{A}) and small, short oligomer C chains (O_{C}); where the ratio of O_{A} oligomers, x, to O_{C}, oligomers, y, is between 1-99%;
vii. a generic (BAB)—O_{A} block copolymer comprising of a longer, linear chain (A), which is di-substituted to two branched polymer components of either a monodisperse or polydisperse structure with 2 or more reactive groups, which in turn are linked to small, short oligomer A chains (O_{A}) and small, short oligomer C chains (O_{C}); where the ratio of O_{A} oligomers, x, to O_{C} oligomers, y, is between 1-99%;
viii. a generic (AB₄)—O_{A})ₓ(O_{C})_{y} block copolymer comprising of a longer, linear chain (A), which is tetra-substituted to four branched polymer components of either monodisperse or polydisperse structure with 2 or more reactive groups which in turn are linked to small, short oligomer A chains (O_{A}) and small, short oligomer C chains (O_{C}); where the ratio of O_{A} oligomers, x, to O_{C} oligomers, y, is between 1-99%;

FIG. 3 shows a thermoformed article made with a blended material whereby the matrix material forms the bulk of the article and the branched material with specific terminal groups has migrated to the surface, with a concentration profile from the bulk to the surface and at the surface of the article:

FIG. 4 is a schematic diagram of a compounder/extruder having mixing screws and a heated plate/die which the molten polymer is forced through, resulting in the final thermoformed article;

FIGS. 5(A)-5(D) illustrate examples of commercially available (from Polymer Factory AB of Sweden) hyperbranched polymers:
Polyester Based Hyperbranched Polymers
   (A) Boltorn U3000 a lipophilic, fatty acid modified dendritic polymer, esterified with 14 unsaturated fatty acids derived from sunflower oil consisting of C16 and/or C18 fatty acids, showing the compact nature of the molecular structure
Polyester Amide Based Hyperbranched Polymers
   (B) Hybrane S1200 built on succinic anhydride and diisopropanol amine with secondary hydroxyl end groups.
   (C) Hybrane D2800 built on dodecenyl succinic anhydride and diisopropanol amine with secondary hydroxyl end groups.
   (D) Hybrane DEO7508500 built on dodecenyl succinic anhydride and diisopropanol amine with polyethylene glycol end groups, with all three materials again presenting a compact molecular structure;

FIG. 6(a) shows a comparison of the chemical composition of the inner surface of three extrusions: (X) a high density polyethylene (HDPE) control extrusion, was compared against (Y) Tube A, an extrusion containing a wt. % of H40 Boltorn hyperbranched polyester and (Z) Tube B, an extrusion with a wt. % of PS2550 Hybrane hyperbranched polyester amine. The analyses was carried out using XPS (X-ray Photoelectron Spectroscopy), a technique which measures the chemical composition within a surface depth of nanometers, whereby the nitrogen in the hyperbranched polyester amine acted like a marker enabling the migration of the hyperbranched additive during the thermoforming process to be tracked;

FIG. 6(b) shows (ii) a further analysis of both the inner and outer surfaces of Tube B (locations on the extruded tubing are indicated in panel (iii)) showing the presence of nitrogen (N) (a constituent of the hyperbranched polyester amine which is not present in HDPE) at both residing surfaces and not in the bulk of the extrusion, (location of which again are indicated in panel (iii)).

FIG. 7(A) is an AFM¹ image, obtained in Tapping Mode, showing a homogeneous, single phase morphology for the pure, extruded HDPE polymer matrix, the Control Tube in FIG. 6(a); ¹ Atomic Force Microscopy (AFM), a microscope without lenses, operates through interaction between a mechanical probe and the surface of the sample. Using Tapping Mode™, it can map the spatial variation in surface elasticity using a method termed as Phase Imaging. This technique can show microstructural properties of polymer blends, based on their relative elasticity and are hence useful for demonstrating the degree of miscibility between polymer blend phases.

FIG. 7(B) is an AFM image, again obtained in Tapping Mode, of a HDPE matrix containing a wt. % of hyperbranched polyester polymer, Tube A in FIG. 6(a), showing a phase separated morphology, where the blends are essentially immiscible or incompatible;

FIG. 8 exhibits a comparison of Ultimate Tensile Strengths (UTS) of a tube/ISO594 luer overmould bond for Control Tube, Tube A, and Tube B (left hand side of the graph) and the inherent UTS of each Tube extrusion, tested to destruction (right hand side of the graph);

FIG. 9(a) shows the building blocks, branching units and end-capping species used to produce the hyperbranched polyester amide, DEO7508500 where the resultant structure of the Hybrane DEO7508500 includes functional hydrophilic methoxy-terminated PEG terminal groups with a hydrophobic core is shown in FIG. 5(D);

FIG. 9(b) shows a comparison of the Differential Scanning calorimetry (DSC) curves for the HDPE control (Marlex virgin HDPE) and DNT750PE (HDPE host matrix with a 6 wt. % of DEO7508500) showing the heat of enthalpy (H) (J/g) and melting temperature (Tₘ) (° C.) for both;

FIG. 10 is a graph which compares the dynamic coefficient of friction (μ_{d}) obtained for compressed sheets ( [symbol] ) made from the control HDPE matrix and DNT750PE (HDPE host matrix with a 6 wt. % of DEO7508500), with extruded sheets ( [symbol] ) made from the control HDPE matrix and DNT750PE (HDPE host matrix with a 6 wt. % of DEO7508500). Each set of samples (i.e.) compressed and extruded, where compared with compressed and extruded polytetrafluoroethylene (PTFE) sheeting, which was tested twice, each time the test was set up. The tests were carried out using the ISO test standard ASTM D1894. The extruded sheets were produced according to the thermoforming extrusion parameters; feed rate and screw speed, exhibited in Table 2;

FIGS. 11(a) and 11(b) show the building blocks, branch species and linear chains used to produce the customised branched hybrid polymer called Factor DNT022;

FIG. 11(c) shows the resultant molecular structure arising from the reaction represented in FIG. 11(b) producing an additive with a core linear PEG chain (in this case 6 k, where 6 k refers to the average molecular weight of the polymer) connected to short PEG oligomer chains via a branched polymer component in the form of a polyester HBP of G3 (G=Generation, the number of consecutive branching points);

FIG. 12(a) shows a ¹³C NMR spectra confirming the absence of free reactant, carboxylic acid functionalised methoxyl polyethylene glycol acid (m-PEG750COOH), the end-capping ingredient or —OH groups in the final DNT022 material, indicating that the branched polymer component (G3) links the terminal PEG oligomers elements;

FIG. 12(b) is a graph showing a comparison of the gel permeation chromatography (GPC) curves confirming peglation of the branched hybrid DNT022 (6kG3);

FIG. 13(a) is a graph which shows the difference in the quantity of the ether (—C—O—C—, a constituent of PEG chains) peak at 1117 cm⁻¹, as measured by Raman Spectroscopy present at the surface of the PEBAX² control compared with DNT022PX sheets. The DNT022PX sheets exhibited a peak 8.4% bigger than that in PEBAX sheets, whilst the same peak increased in intensity by 21% following immersion in deionised water; ² PEBAX Polyether block amide is a thermoplastic elastomer made of flexible polyether and rigid polyamide. The non-limiting grade chosen in this example (7233 SA 01 MED) is specially designed to meet the stringent requirements of the medical applications such as minimally invasive devices.

FIG. 13(b) is a graph which shows the dynamic coefficient of friction (μ_{d}) (according to ASTM D1894-11) of the sheets measured following immersion in deionised water, demonstrating a μ_{d} for DNT022PX lower than that obtained for both the PEBAX control and PTFE sheets.

FIG. 14 is a graph which shows size exclusion chromatography (SEC) data for a starter material of a larger methoxyl polyethylene glycol acid (m-PEG750COOH (PEG20k-G3-OH)) and the final material (PEG-20k-G3-PEG) showing an expected increase in molecular weight at the end of the reaction, again confirming, the conversion of —OH groups to -MPEG groups. This data, together with the information presented in FIGS. 12(a)-12(b), indicates the ability to convert —OH terminal groups to -MPEG groups for a range of linear-dendritic hyperbranched polymers;

FIG. 15(a) presents a theoretical conformational structure of a Boltorn H₂O P with a 4 functional core connected to G3 hyperbranched structures;

FIG. 15(b) shows the corresponding theoretical molecular structure of Boltorn H₂O P;

FIG. 15(c) presents a theoretical conformational structure of a pegylated linear dendritic (L-D) hyperbranched PEG attached to linear 2 functional cores connected to hyperbranched structures with various potential generations. Comparing FIGS. 15(a) and 15(c) demonstrates the compact nature of the Boltorn based hyperbranched polymer verses the larger L-D hyperbranched structure;

FIG. 15(d) shows the corresponding theoretical molecular structure of two pegylated L-D hyperbranched PEG additives comprising of a linear core of Xk length connected to two hyperbranched structures with two functional cores each, extending to a generation number of 3 or 5. These images illustrate the differences in size of various additive combinations;

FIG. 16(a) is a size exclusion chromatography (SEC) graph for PEG 10kG3;

FIG. 16(b) is a size exclusion chromatography (SEC) graph for PEG 6kG5;

FIG. 16(c) is a size exclusion chromatography (SEC) graph for Boltorn H20P;

FIG. 17(a) is a graph which shows a comparison of both static coefficient of friction (μ_{d}) ( [symbol] ) and dynamic coefficient of friction (μ_{d}) ( [symbol] ) for PEBAX Control, modified 6kG3PX sheet (<5 wt. %) and modified 6kG5PX sheet (<5 wt. %);

FIG. 17(b) is a graph which compares the dynamic coefficient of friction (μ_{d}) results of PTFE and PEBAX 7233 control with <5% additions of Boltorn H20 P, 10kG3 and 10kG5 to a PEBAX host matrix;

FIG. 17(c) shows both μₛ ( [symbol] ) and μ_{d} ( [symbol] ), obtained for an extruded control PEBAX 7223 sheet and each of the listed additives in a PEBAX 7233 sheet at <5%, tested according to ASTM D1894-11 with water are presented;

FIG. 18(a) is a graph which shows further evidence of the influence of the length of the core linear chain, across a range from 6 k, 10 k to 20 k, for both G3 ( [symbol] ) and G5 ( [symbol] ) based linear dendritic hyperbranched additives in the PEBAX 7233 host matrix on the static coefficient of friction (μₛ) compared to that obtained for the PEBAX 7233 virgin control and PTFE;

FIG. 18(b) similarly demonstrates the influence of length of the core linear chain for both G3 ( [symbol] ) and G5 ( [symbol] ) based linear dendritic hyperbranched additives in a PEBAX 7233 host matrix on the dynamic coefficient of friction (μ_{d}) compared to the PEBAX 7233 host virgin control matrix and PTFE;

FIG. 19(a) is a bar chart which compares the results of elution tests conducted on control HDPE and PEBAX extruded sheets to HDPE and PEBAX extruded sheets with hyperbranched additives using human dermo fibroblasts; and

FIG. 19(b) is a bar chart which compares the results of elution tests conducted with control HDPE and PEBAX extruded sheets to HDPE and PEBAX extruded sheets with hyperbranched additives using mouse macrophage.

### DETAILED DESCRIPTION

The particular values and configurations discussed in these non-limiting examples can be varied and are cited merely to illustrate embodiments of the disclosed compositions and methods and are not intended to limit the scope of the disclosure.

A molecule is made up of a group of atoms bonded together, representing the smallest fundamental unit of a chemical compound that can take part in a chemical reaction. A monomer is a molecule that can bind chemically to other molecules to form long chains called polymers. In general, linear polymers can comprise two terminal end groups with a repeating unit between the ends, with an oligomer comprising a molecule of intermediate relative molecular mass, the structure of which essentially comprises a small plurality of units derived, actually or conceptually, from molecules of lower relative molecular mass. Branching on a linear polymer occurs by the replacement of a substituent, (e.g.) a hydrogen atom, on a monomer sub-unit, by another covalently bonded chain of that polymer; or, in the case of a graft copolymer, by a chain of another type.

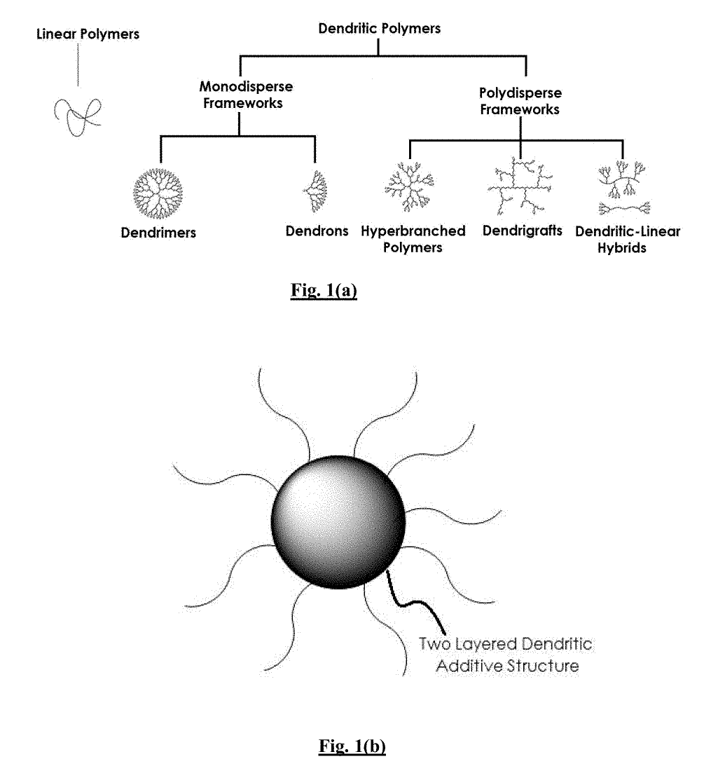

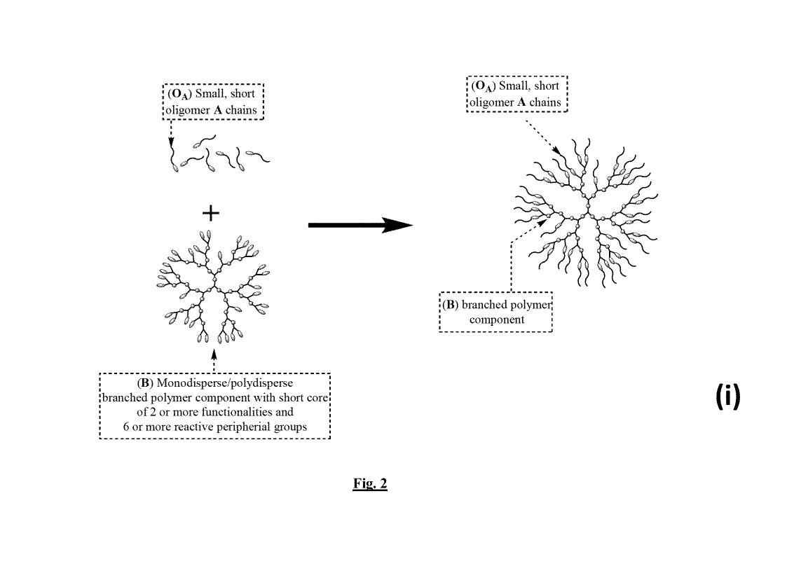

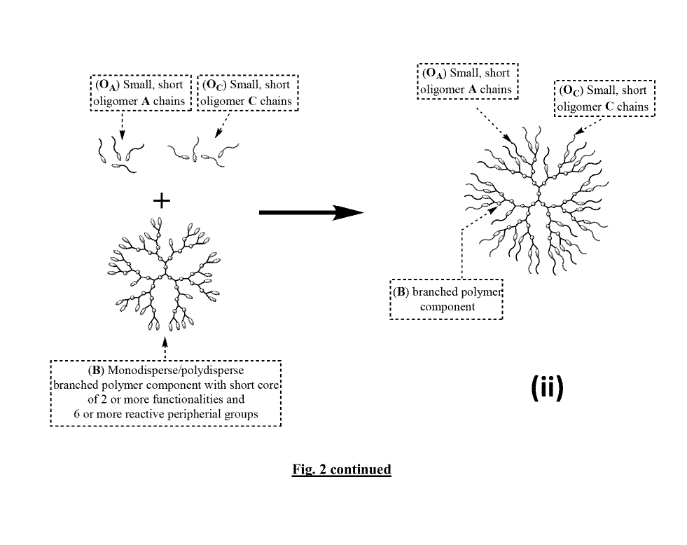

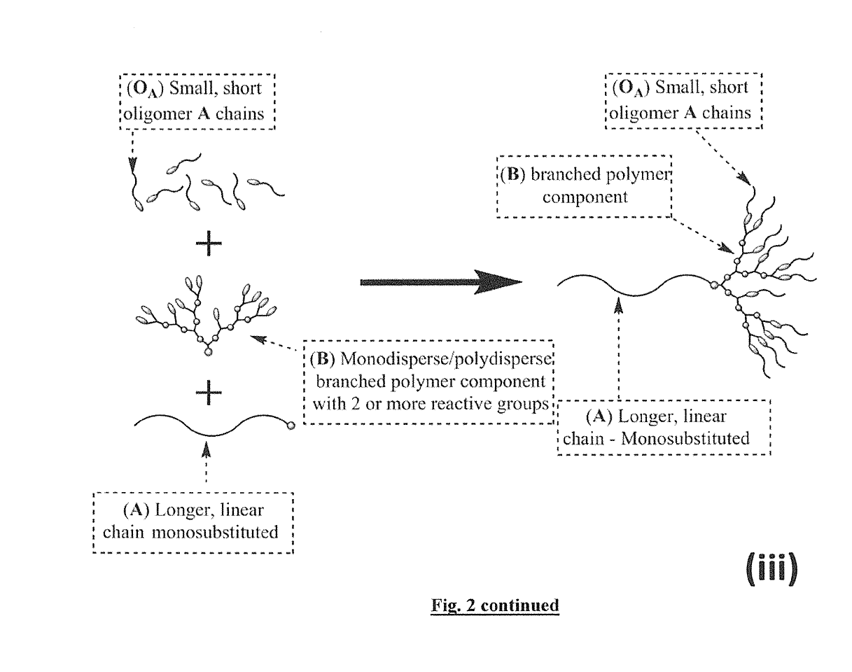

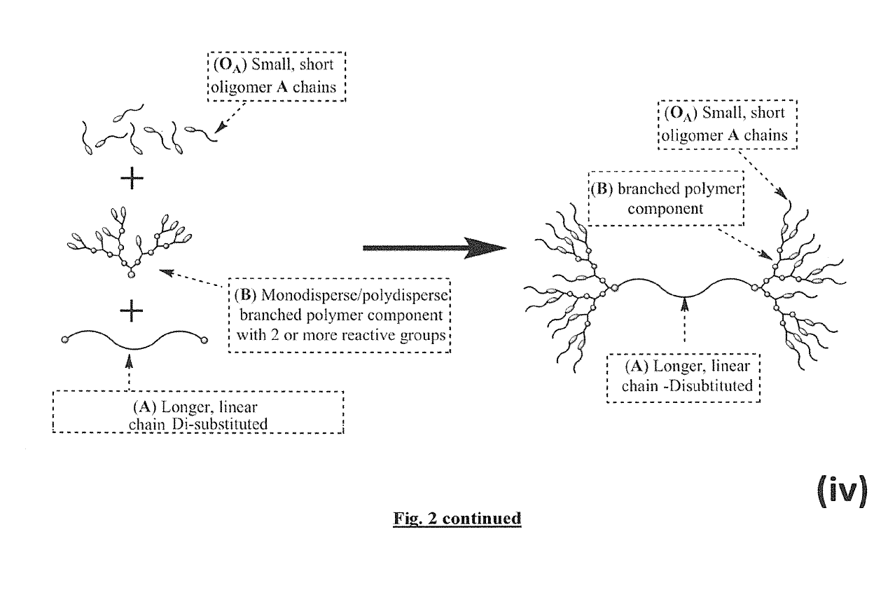

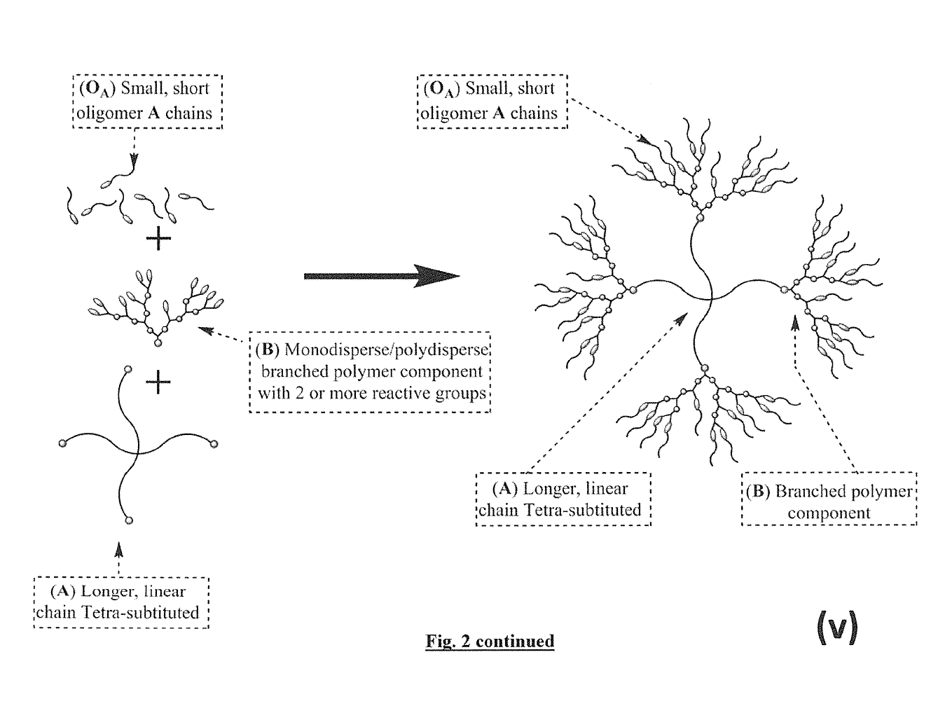

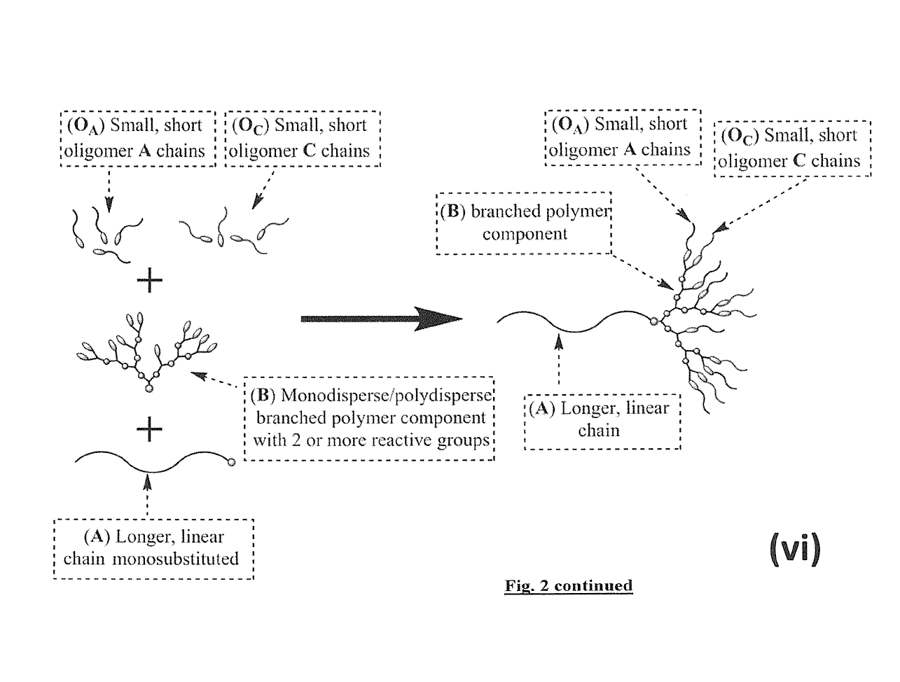

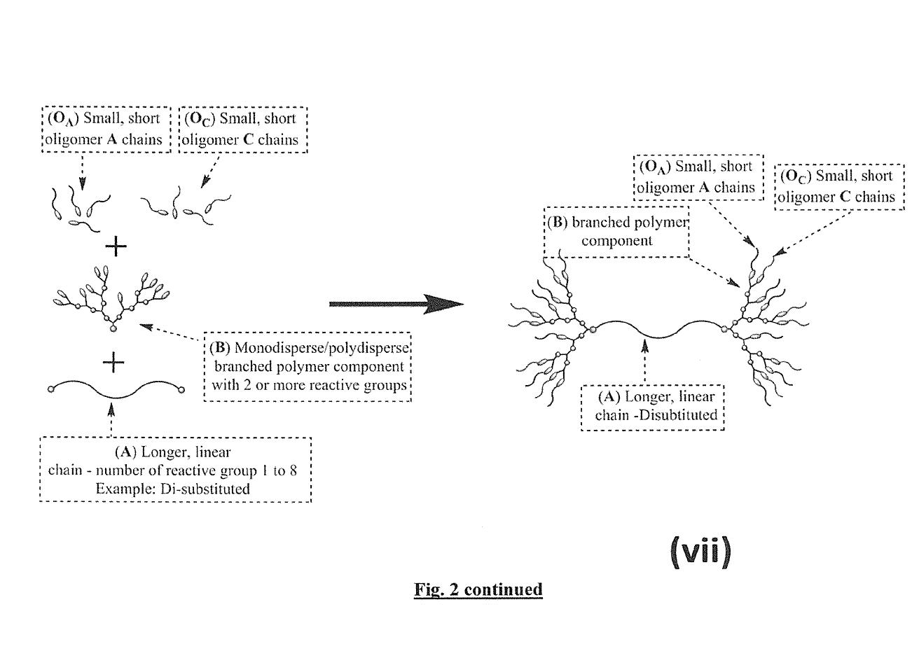

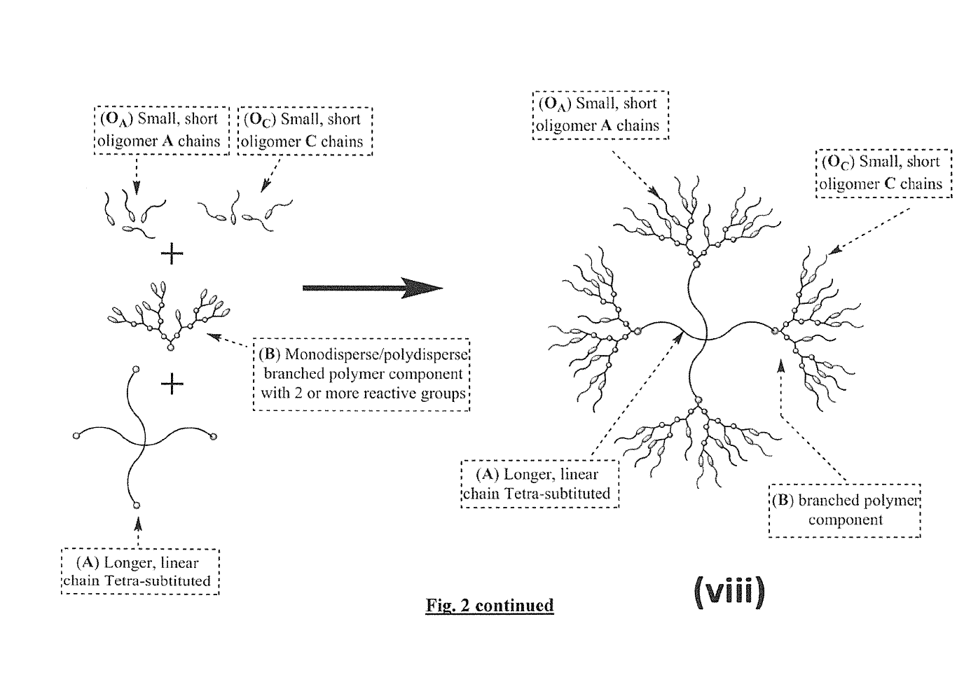

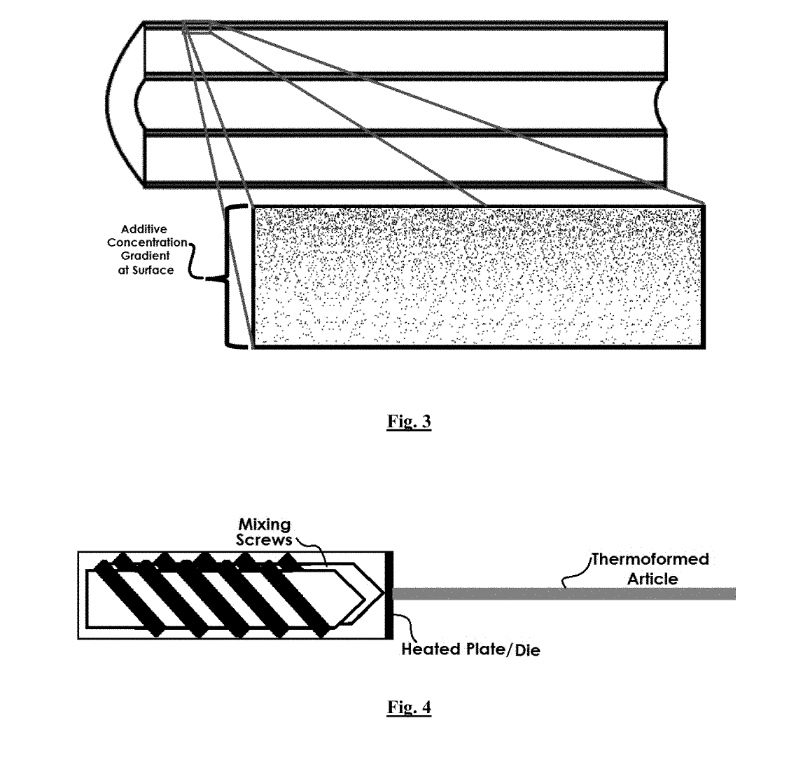

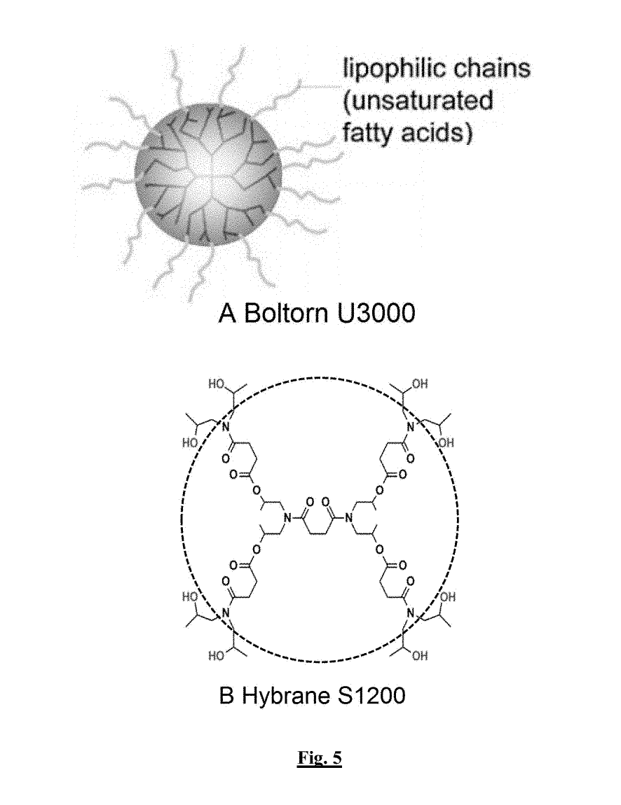

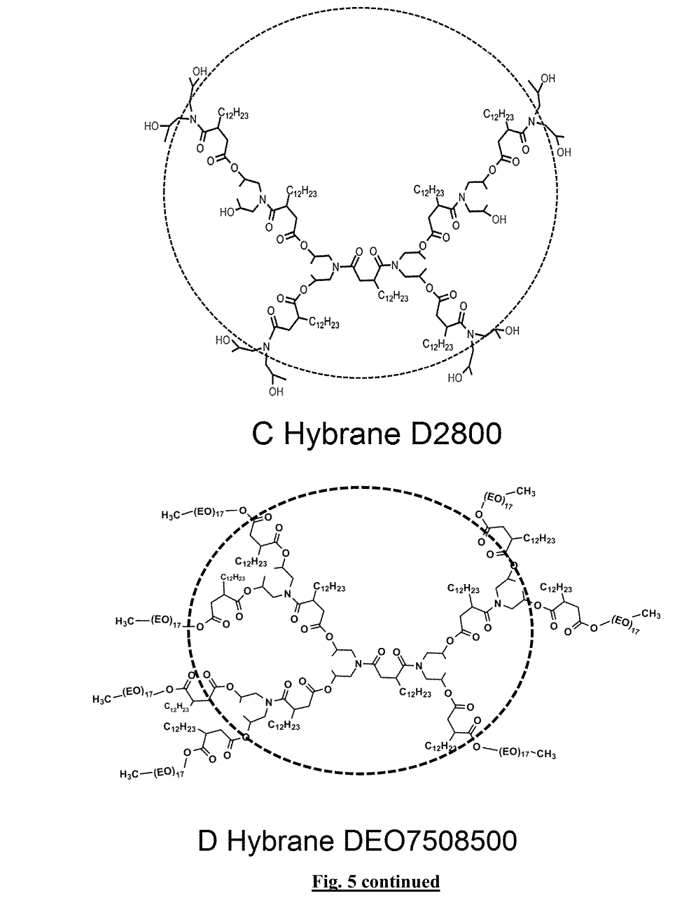

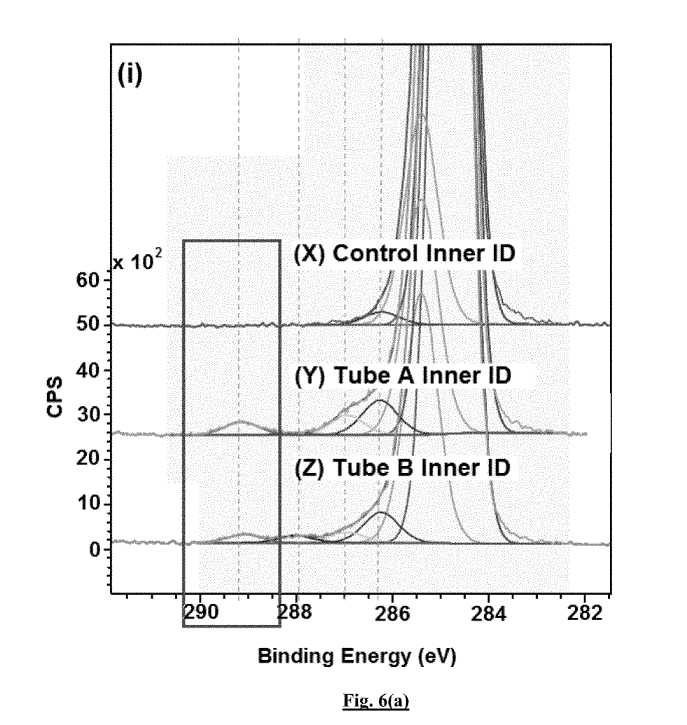

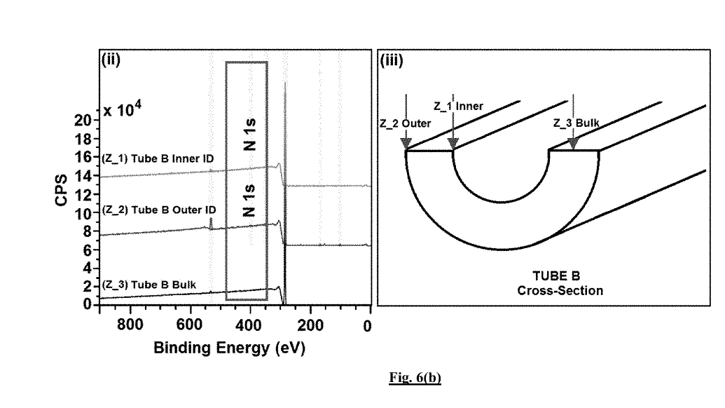

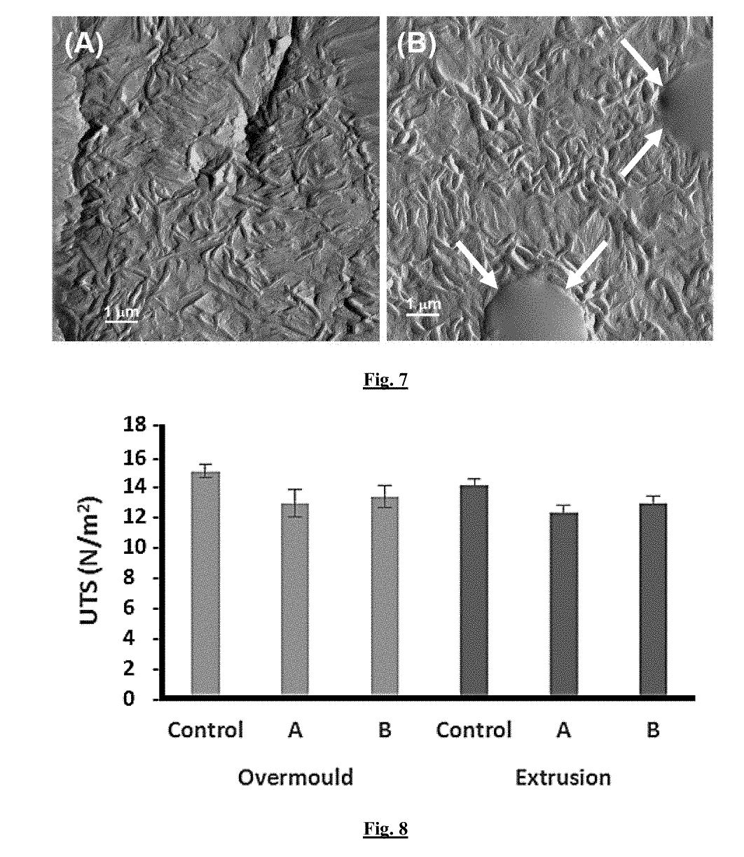

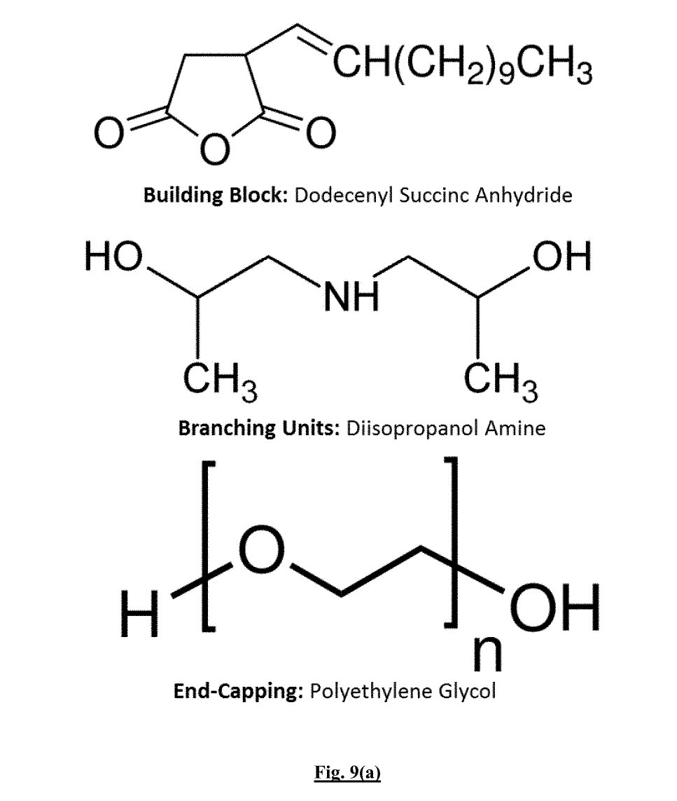

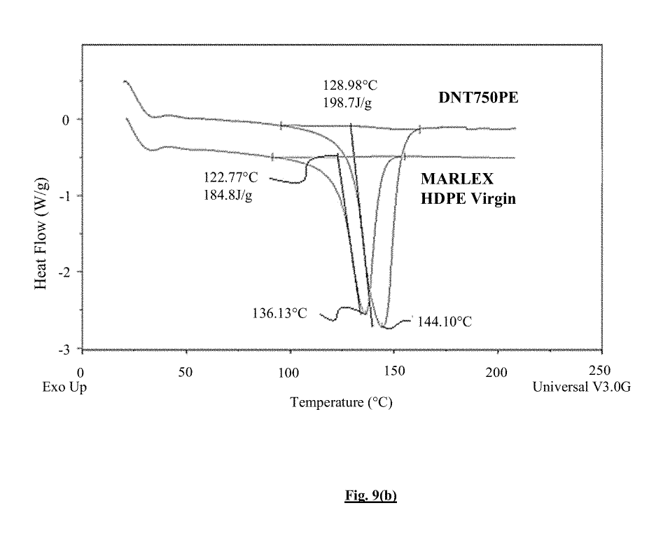

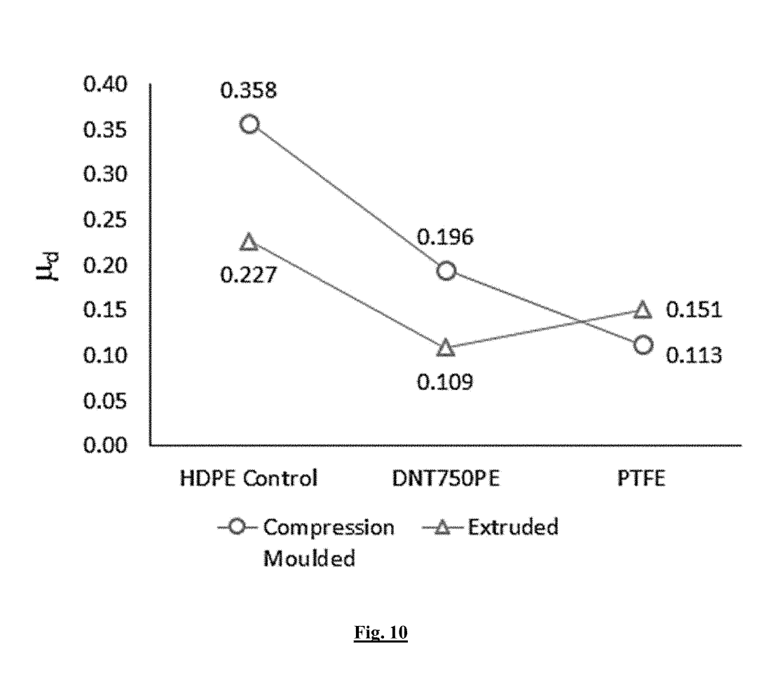

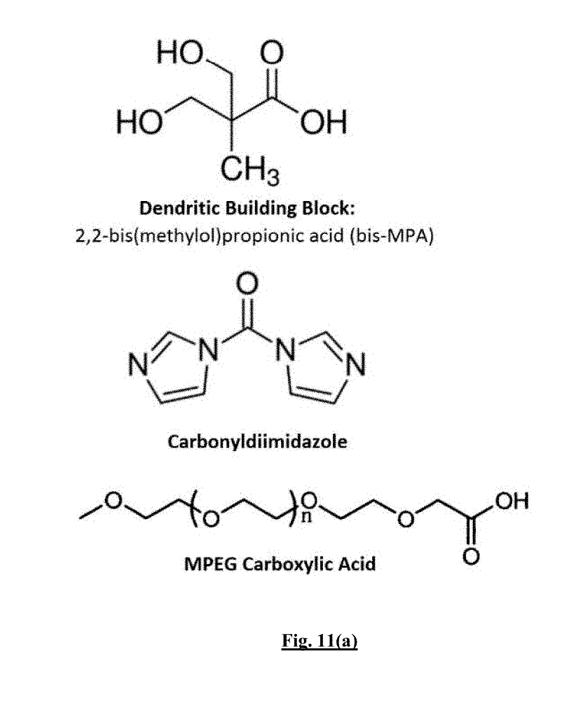

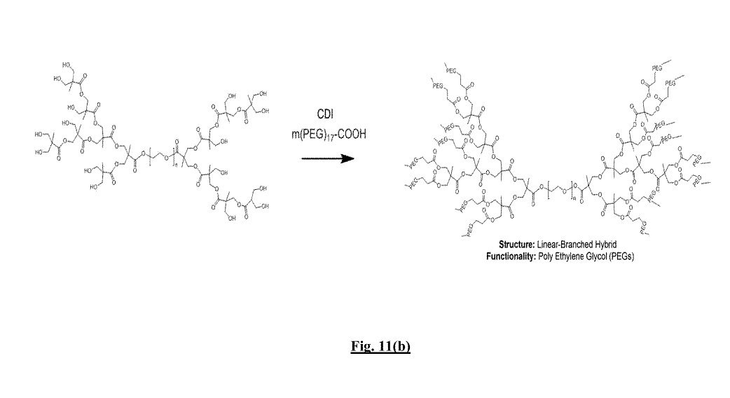

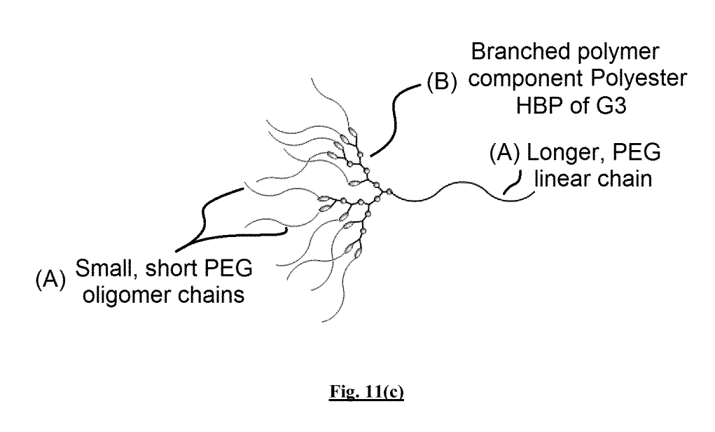

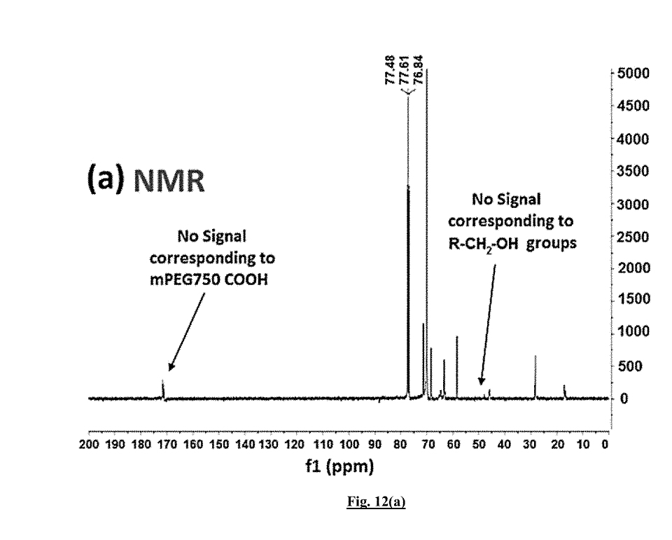

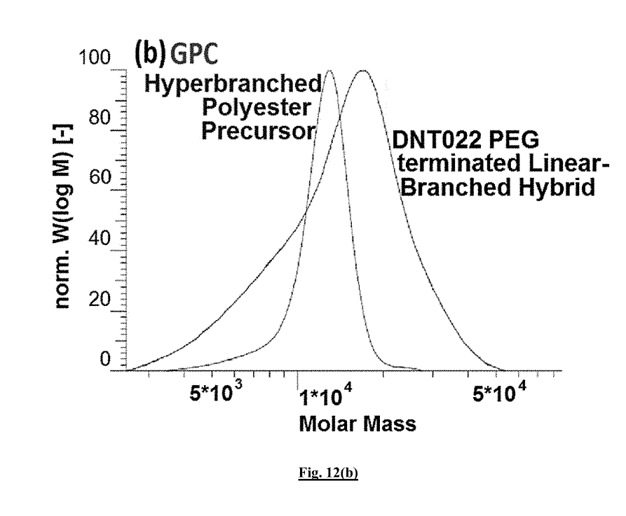

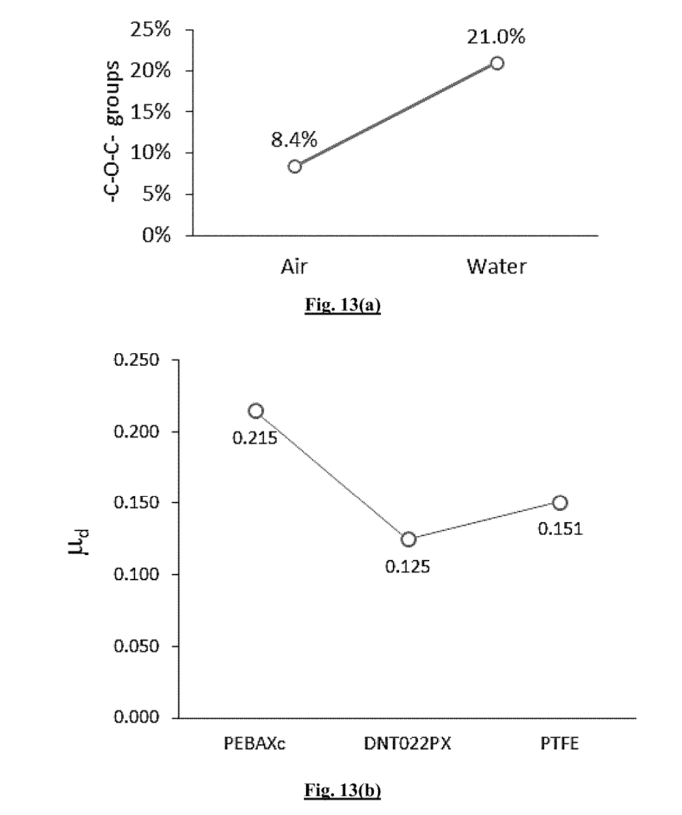

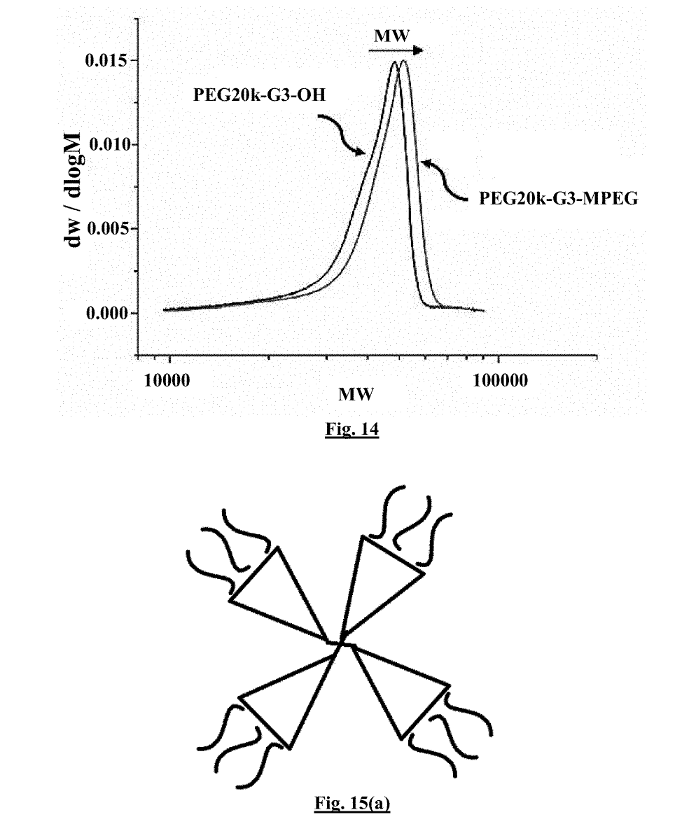

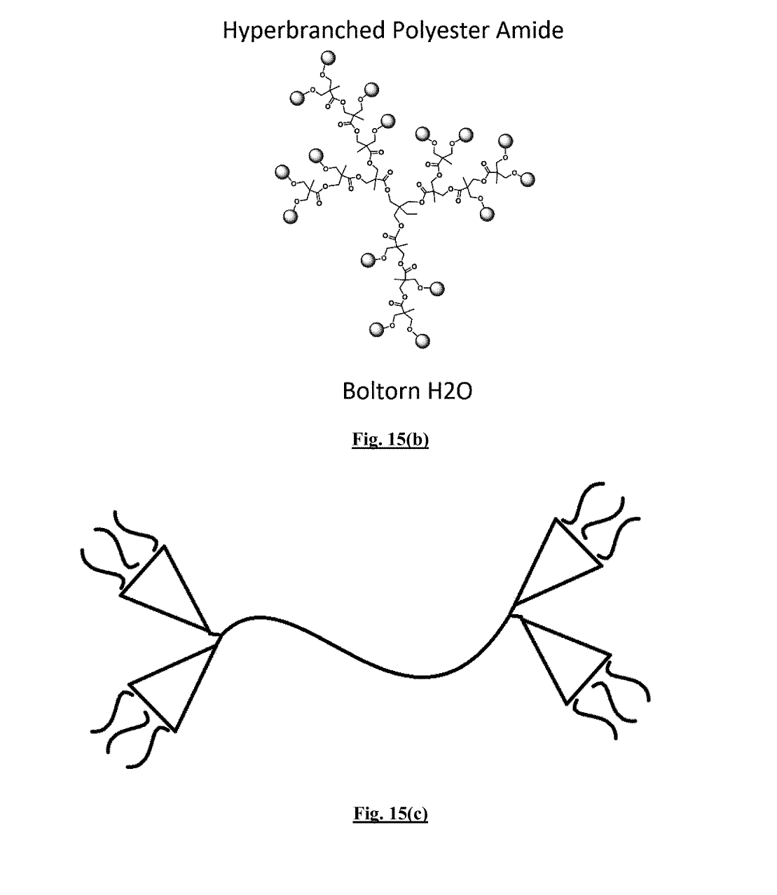

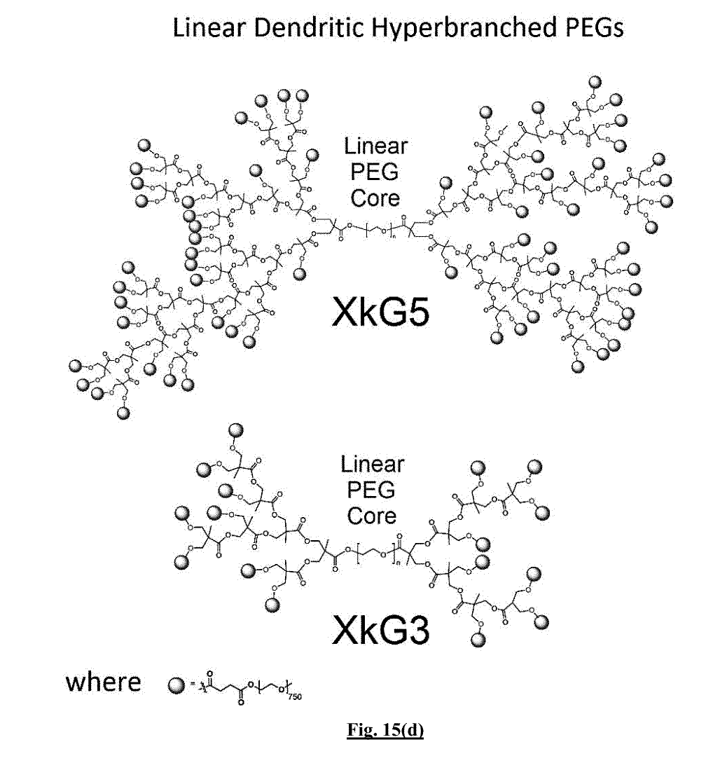

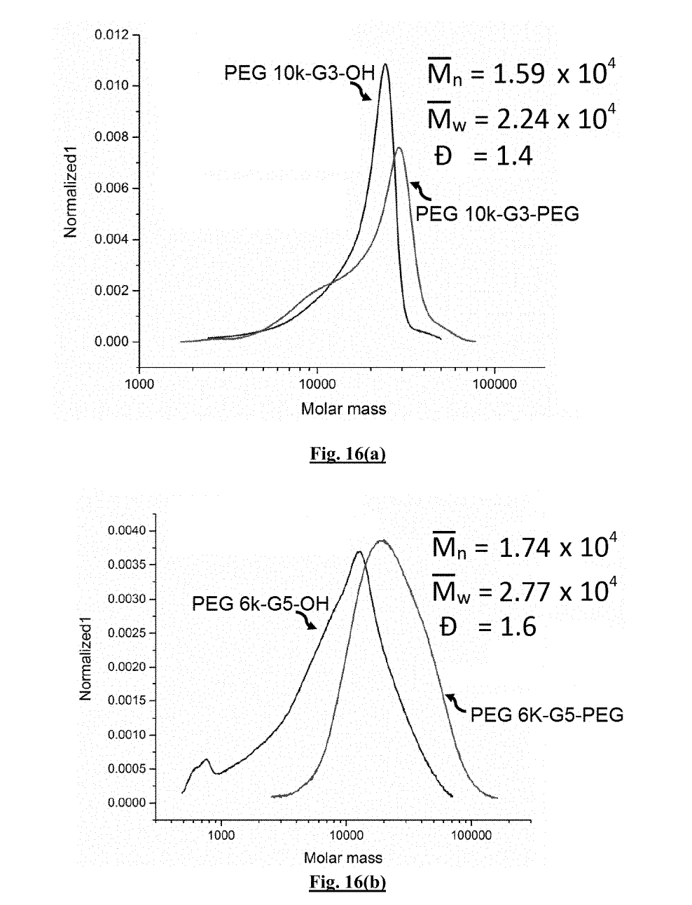

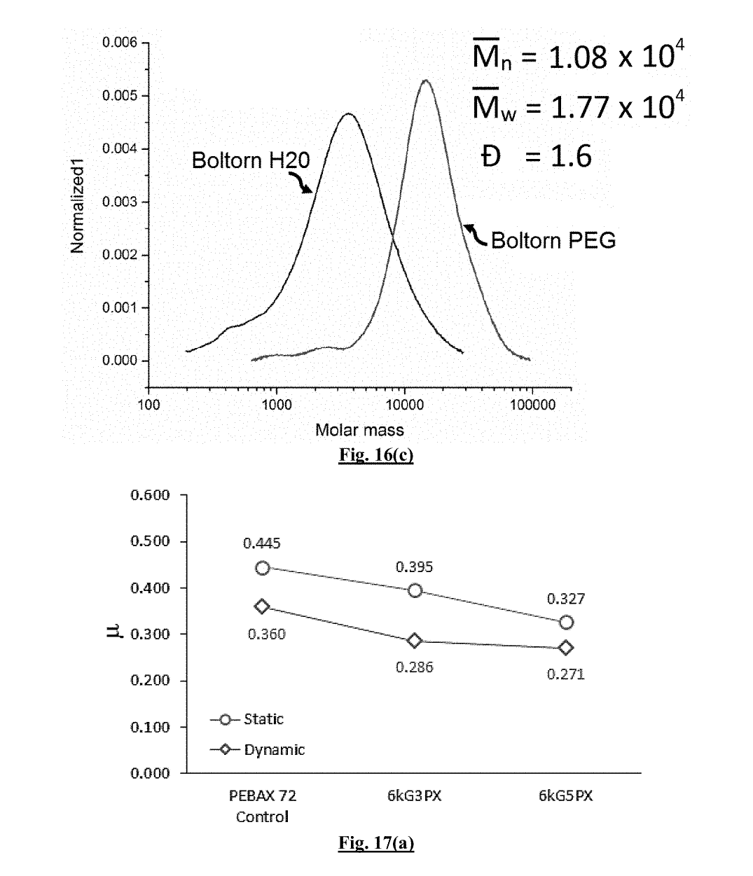

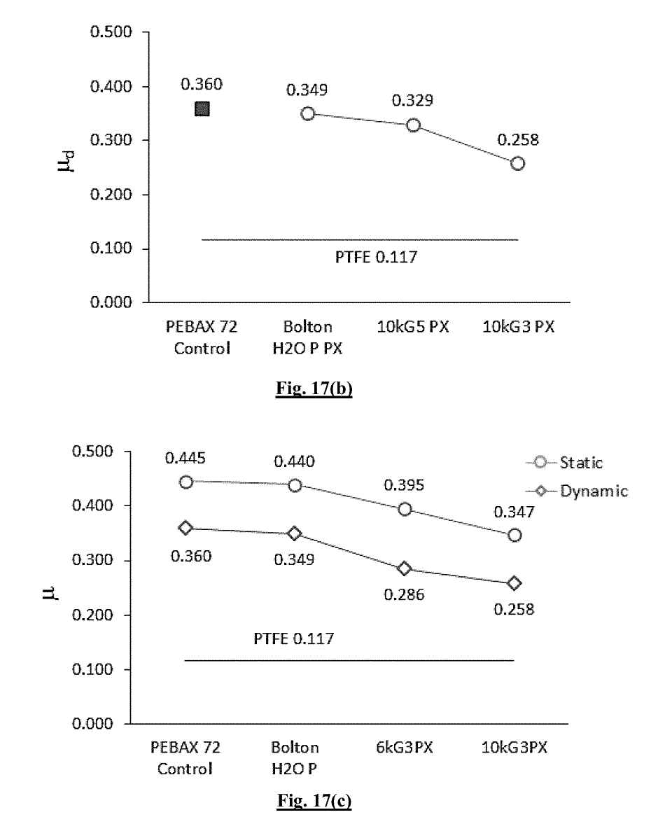

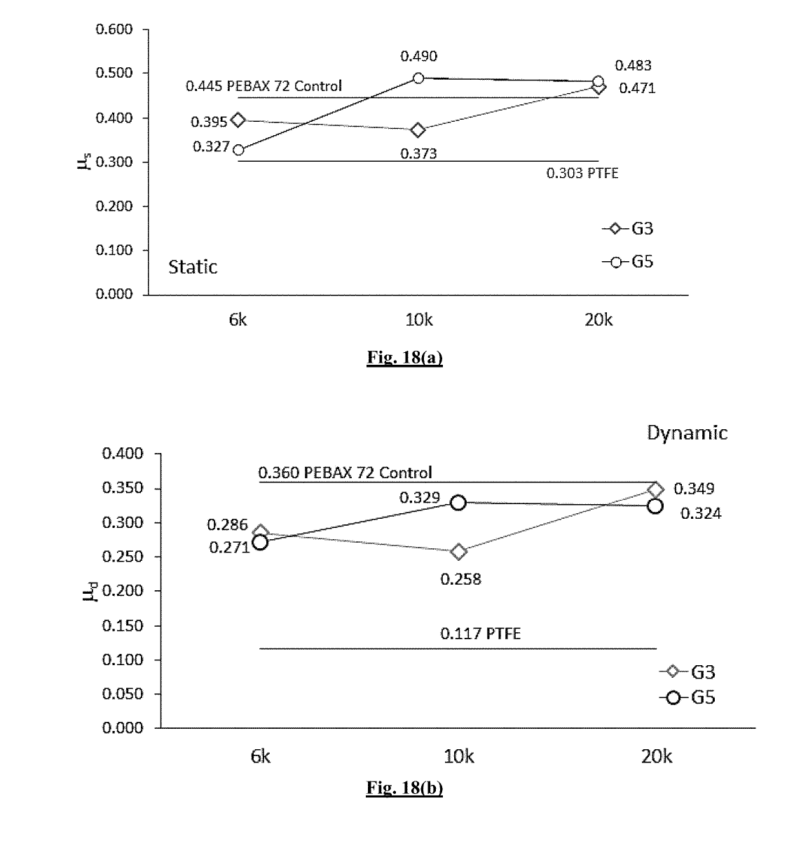

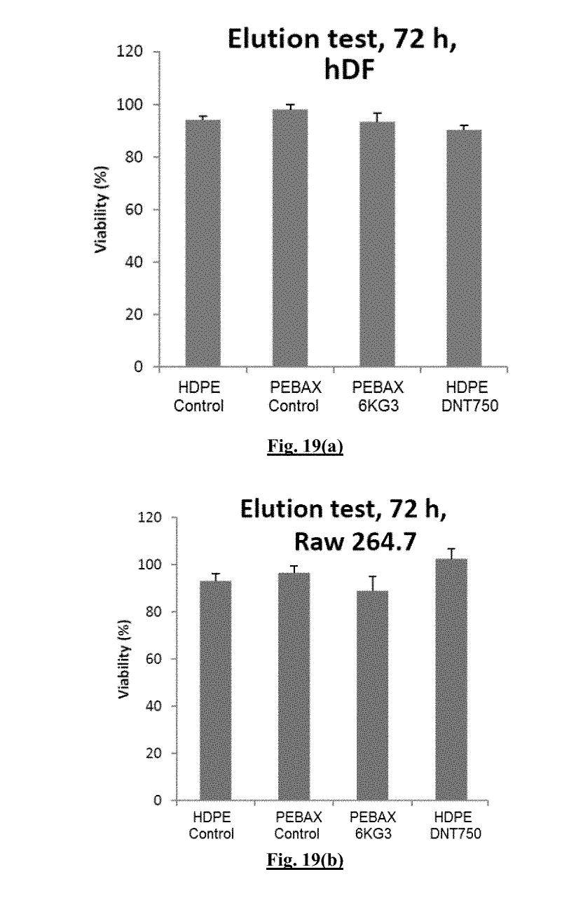

Branching is also employed in dendritic polymers, where the structure comprises a core surrounded by at least two or a larger number of monomers that branch outwards. Dendritic polymers are typically split into two distinct categories based on the perfection or otherwise of their structure, as illustrated in FIG. 1(a). Monodisperse frameworks are perfect structures (perfectly symmetrical) and include dendrimers and dendrons. Polydisperse frameworks are made up of imperfect structures (not perfectly symmetrical), like hyperbranched polymers, dendigraphs and linear-dendritic hybrids, which are by definition block co-polymers.

The periphery or outer shell of these dendritic structures comprises of multiple small reactive groups that can be post-modified with specific substituents, which may provide a desired property to the branched polymer. Due to the multiple representation of peripheral groups the post functionalisation enables the design of a dendritic polymer that exhibits intrinsically different properties from the unmodified pre-polymer. The final property of a dendritic material is reflected by its building blocks i.e. core, monomers and peripheral groups. In its essence the dendritic polymer is a compact, soft nanoparticle polymer that is highly branched, and unlike linear polymers, which are subjected to inter- and intramolecular entanglements due to their random coil conformation, dendritic polymer typically experience little to no entanglements. By carefully selecting the core, the set of monomers as well as peripheral groups, a layered branched polymer is achieved.

Polyester hyperbranched polymers are known and are commercially available under the brand name Boltorn. Hybranes are commercially available hyperbranched polyesteramides. The generic structure for both is shown in FIG. 1(b), having a central hydrophobic core with many terminal end groups. Such polymers have been used to modify the surface of moulded parts.

Dendrimers typically exhibit layered and heterogeneous properties with core-shell features, which adapt conformation to the surrounding environment. When this environment is within a bulk polymer matrix, the inherent properties of the dendritic structure, coupled with its constrained geometry and lack of entanglements allow it to act as a mode of transportation adapting to its environs as it migrates. This constrained structural conformation aids in its travel through the host polymer matrix as it has less obstacles to compete with when compared to a linear polymer, which is subjected to both random coil configuration and entanglement in the bulk polymer matrix. Additionally, polar heterogeneity between the host polymer matrix and the branched hybrid polymer also may influence the migration of the branched hybrid polymer through the host polymer. As this incompatibility increases in magnitude, the concentration gradient of the branched polymer additive at the host polymer matrix surface/air interface is expected to increase.

A branched polymer with short cores of a functionality of 2 or more and 6 or more reactive peripheral groups linked to oligomer chains or core linear polymers linked to smaller oligomers via branched components producing an (A)(B)(A) structure have not previously been described. The branched polymer component may include either a monodispersed dendritic polymer or polydispersed hyperbranched polymer. Aspects of the present disclosure include reacting such branched polymer components with linear polymers and a plurality of oligomers to produce a branched polymer hybrid of structure (A)(B)(A). The linear and oligomer chains are selected in order to achieve a desired property at the surface of the final solid article, e.g., final solid polymer substrate.

Accordingly aspects of this invention include a platform branched hybrid polymer (components of which include linear polymer chains, small oligomer chains, and branched polymers of either a polydispersed hyperbranched or a monodispersed dendritic nature), which is in turn blended in a host polymer. These platform branched hybrid polymers may be utilized as a vehicle to transport specific, small, functionalized chains of the branched hybrid polymers to the surface/air interface of the thermoformed. matrix polymer article, delivering a concentration gradient that may provide a selected surface property to the thermoformed article as diagrammatically illustrated in FIG. 3.

The terms “host polymer,” “matrix polymer” and “host polymer matrix” as detailed herein means a polymer that forms the bulk constituent of the thermoformed article.

The term “linear polymer” detailed herein means a polymer having a linear chain structure or backbone. In at least one example, the linear polymer may be selected from the group consisting of:
poly(methylacrylate)s, polyesters, poly(alkylene diol)s, poly(alkylene diol monoalkyl ethers, poly(aryl ether)s, poly(vinyl alcohol)s, poly(acrylamide)s, poly(urea)s, poly(urethane)s, poly(methacrylamide)s, poly(ethylene imine)s, poly(ethylene glycol), poly(vinyl ether)s, poly(vinyl ester)s, poly(epichlorohydrin), poly(glycidyl ether)s, poly(glycidyl ester)s, poly(carbonate)s, poly(thio ether)s, poly(thio ester)s, polyamides, epoxy resins, novalac resins and quaternary ammonium polyacrylates and polyamines; succinc anhydrides, triglycerides and saturated fatty acids, for example, but not limited to, behenic/docosanoic acid, palmitic acid, stearic acid, silicon based polymer compounds, for example, but not limited to poly(dimethyl siloxane) (PDMS) and fluorine based polymer compounds, for example, but not limited to polytetrafluoroethylene (PTFE), ethylene tetrafluoroethylene (ETFE), perfluoroalkoxy alkane (PFA), perfluorooctanoic acid (PFOA), polyvinylidene fluoride (PVDF), hexafluoropropylene (HFP), and combinations thereof.

The term “oligomer” as detailed herein refers to the IUPAC definition of:
An oligomeric or polymeric offshoot from a macromolecular chain.

Notes:
1. An oligomeric branch may be termed a short-chain branch.
2. A polymeric branch may be termed a long-chain branch.
and detailed herein is an oligomer selected from the group consisting of:
poly(methylacrylate)s, polyesters, poly(alkylene diol)s, poly(alkylene diol monoalkyl ethers, poly(aryl ether)s, poly(vinyl alcohol)s, poly(acrylamide)s, poly(urea)s, poly(urethane)s, poly(methacrylamide)s, poly(ethylene imine)s, poly(ethylene glycol), poly(vinyl ether)s, poly(vinyl ester)s, poly(epichlorohydrin), poly(glycidyl ether)s, poly(glycidyl ester)s, poly(carbonate)s, poly(thio ether)s, poly(thio ester)s, polyamides, epoxy resins, novalac resins and quaternary ammonium polyacrylates and polyamines; succinc anhydrides, triglycerides and saturated fatty acids, for example, but not limited to, behenic/docosanoic acid, palmitic acid, stearic acid, silicon based polymer compounds, for example, but not limited to poly(dimethyl siloxane) (PDMS) and fluorine based polymer compounds, for example, but not limited to PTFE, ETFE, PFA, PFOA, PVDF, HFP, and combinations thereof.

The term “branched polymer component” as detailed herein refers to:
“hyperbranched polymers” meaning polydispersed frameworks containing chain architectures with multiple branches, with many terminal groups, joined together in a compact but irregular (non-symmetrical) way.
“dendrimer” meaning monodispersed frameworks containing a chain architecture that is perfectly (symmetrically) branched, having a tree-like structure, usually more than 2 generations. The generation of a branched polymer refers to the number of consecutive branching points. For example, a third generation branched polymer refers to a polymer with three consecutive branching points.

The terms “active agents” and “bound agents” detailed herein include adjuvants selected from the group consisting of:
antimicrobial agents (which may be bound to a dendritic polymer) including but not limited to zinc oxide compounds, silver compounds, benzimidazole derivatives, hydrochloric acid, a taurinamide derivative, a phenol, quaternary ammonium surfactant, chlorine-containing, quinoline, quinaldinium, lactone, dye, thiosemicarbazone, quinone, sulfa, carbamates, urea, salicylamide, carbanilide, amide, guanide, amidine, chelate, imidazoline biocides, acetic acid, benzoic acid, sorbic acid, propionic acid, boric acid, dehydroacetic acid, sulfurous acid, vanillic acid, esters of p-hydroxybenzoic acid, ethanol, isopropanol, propylene glycol, benzyl alcohol, chlorobutanol, phenylethyl alcohol. 2-bromo-2-nitropropan-1,3-diol, formaldehyde, glutaraldehyde, calcium hypochlorite, potassium hypochlorite, sodium hypochlorite, iodine (in various solvents), povidone-iodine, hexamethylenetetramine, noxythiolin, 1-(3-choroallyl)-3,5,7-triazo 1-azoniaadamantane chloride, taurolidine, taurultam, EDTA, N(5-nitro-2-furfurylidene)-1-amino-hydantoin, 5-nitro-2-furaldehyde semicarbazone, 3,4,4′-trichlorocarbanilide, 3,4′,5-tribromosalicylanilide, salicylanilide, 3-trifluoromethyl-4,4′-dichlorocarbanilide, 8-hydroxyquinoline, 1-cyclopropyl-6-fluoro-1,4-dihydro-4-oxo-7-(1-piperazinyl)-3-quinolinecarboxylic acid, 1,4-dihydro-1-ethyl-6-fluoro-4-oxo-7-(1-piperazinyl)-3-quinolinecarboxylic acid, hydrogen peroxide, peracetic acid, phenol, sodium oxychlorosene, parachlorometaxylenol, 2,4,4′-trichloro-T-hydroxydiphenol, thymol, chlorhexidine, benzalkonium chloride, cetylpyridinium chloride, silver, nanosilver, silver sulfadiazine, silver nitrate, 5 fluorouracil, phenolic antiseptics, gentian violet, methylene blue, brilliant green, and bismuth compounds;
anticoagulant agents including but not limited to di-ammonium hydrogen citrate, di-ammonium tartrate, N-(2-bis(carboxymethyl)aminoethyl)-N-(2-hydroxyethyl)glycin salt dihydrate, citric acid, citric acid disodium salt, citric acid monopotassium salt, citric acid monosodium salt, citric acid tripotassium salt, citric acid trisodium salt, ethylenediaminetetraacetic acid (EDTA), EDTA diammonium salt, EDTA dipotassium salt, EDTA disodium salt, EDTA tetrasodium salt, ethylenebis(oxyethylenenitrilo)tetraacetic acid (EGTA), EDTA trisodium salt, EDTA tripotassium salt, ethylene glycol-O,O-bis(2-aminoethyl)-N,N,N,N-tetraacetic acid, N-(2-hydroxyethyl)ethylenediamine-N,N,N-triacetic acid trisodium salt, nitrilotriacetic acid, potassium sodium tartrate, potassium hydrogen D-tartrate, L-tartaric acid dipotassium salt, L-tartaric acid disodium salt, L-tartaric acid monosodium salt, tris(carboxymethyl)amine, heparin, warfarin, acetylsalicylic acid, ibuprofen, indomethacin, prostaglandins, sulfinpyrazone, streptokinase, urokinase, tissue plasminogen activator, coumarin, protamine sulfate, anti-thrombin III, Coumadin, protein C/protein S, nicoumalone, phenprocoumon, hirudin, hirulog, glycosaminoglycans, and mixtures of the foregoing; antibacterial agents that interfere with the growth and reproduction of bacteria, with a function of disinfecting, surfaces and eliminating potentially harmful bacteria; or
antibiotic agents with the function of treating bacterial infections.

The term “loaded agents” detailed herein means either
antimicrobial agents bound to a dendritic polymer including but not limited to zinc oxide compounds, silver compounds, benzimidazole derivatives, hydrochloric acid, a taurinamide derivative, a phenol, quaternary ammonium surfactant, chlorine-containing, quinoline, quinaldinium, lactone, dye, thiosemicarbazone, quinone, sulfa, carbamates, urea, salicylamide, carbanilide, amide, guanide, amidine, chelate, imidazoline biocides, acetic acid, benzoic acid, sorbic acid, propionic acid, boric acid, dehydroacetic acid, sulfurous acid, vanillic acid, esters of p-hydroxybenzoic acid, ethanol, isopropanol, propylene glycol, benzyl alcohol, chlorobutanol, phenylethyl alcohol, 2-bromo-2-nitropropan-1,3-diol, formaldehyde, glutaraldehyde, calcium hypochlorite, potassium hypochlorite, sodium hypochlorite, iodine (in various solvents), povidone-iodine, hexamethylenetetramine, noxythiolin, 1-(3-choroallyl)-3,5,7-triazo 1-azoniaadamantane chloride, taurolidine, taurultam, EDTA, N(5-nitro-2-furfurylidene)-1-amino-hydantoin, 5-nitro-2-furaldehyde semicarbazone, 3,4,4′-trichlorocarbanilide, 3,4′,5-tribromosalicylanilide, salicylanilide, 3-trifluoromethyl-4,4′-dichlorocarbanilide, 8-hydroxyquinoline, 1-cyclopropyl-6-fluoro-1,4-dihydro-4-oxo-7-(1-piperazinyl)-3-quinolinecarboxylic acid, 1,4-dihydro-1-ethyl-6-fluoro-4-oxo-7-(1-piperazinyl)-3-quinolinecarboxylic acid, hydrogen peroxide, peracetic acid, phenol, sodium oxychlorosene, parachlorometaxylenol, 2,4,4′-trichloro-2′-hydroxydiphenol, thymol, chlorhexidine, benzalkonium chloride, cetylpyridinium chloride, silver, nanosilver, silver sulfadiazine, silver nitrate, 5 fluorouracil, phenolic antiseptics, gentian violet, methylene blue, brilliant green, and bismuth compounds;
anticoagulant agents including but not limited to di-ammonium hydrogen citrate, di-ammonium tartrate, N-(2-bis(carboxymethyl)aminoethyl)-N-(2-hydroxyethyl)glycin salt dihydrate, citric acid, citric acid disodium salt, citric acid monopotassium salt, citric acid monosodium salt, citric acid tripotassium salt, citric acid trisodium salt, ethylenediaminetetraacetic acid (EDTA), EDTA diammonium salt, EDTA dipotassium salt, EDTA disodium salt, EDTA tetrasodium salt, ethylenebis(oxyethylenenitrilo)tetraacetic acid (EGTA), EDTA trisodium salt, EDTA tripotassium salt, ethylene glycol-O,O-bis(2-aminoethyl)-N,N,N,N-tetraacetic acid, N-(2-hydroxyethyl)ethylenediamine-N,N,N-triacetic acid trisodium salt, nitrilotriacetic acid, potassium sodium tartrate, potassium hydrogen D-tartrate, L-tartaric acid dipotassium salt, L-tartaric acid disodium salt, L-tartaric acid monosodium salt, tris(carboxymethyl)amine, heparin, warfarin, acetylsalicylic acid, ibuprofen, indomethacin, prostaglandins, sulfinpyrazone, streptokinase, urokinase, tissue plasminogen activator, coumarin, protamine sulfate, anti-thrombin III, Coumadin, protein C/protein S, nicoumalone, phenprocoumon, hirudin, hirulog, glycosaminoglycans, and mixtures of the foregoing;
antibacterial agents that interfere with the growth and reproduction of bacteria, with a function of disinfecting surfaces and eliminating potentially harmful bacteria; or
antibiotic agents with the function of treating bacterial infections.

The step of blending the additive with the host polymer prior to thermoforming may, for example, be selected from the group consisting of mixing, melt blending, solution blending, and/or mixing said host polymer with said additive in a mutual solvent followed by dispersion blending and extrusion compounding.

Though the thermoforming of the present invention is not particularly limited, it may be effected by extrusion forming, multilayer extrusion forming, profile extrusion forming and the like means, utilising either a twin or single screw and a die through which the molten polymer composition is forced to form a continuous shaped article or product. Press forming and vacuum press forming may also be utilised to produce specially formed products, whereby the polymer in a solid form is formed under pressure. In contrast, injection moulding is a manufacturing process for producing parts by injecting material into a mould. Crystalline based polymers may be formed at temperatures approximately in the region of 10 to 40° C. above of their respective melting points, whilst amorphous materials may be formed at approximate temperatures in the region of 80 to 150° C. above of their respective glass transition temperature range. The product produced from each of these thermoforming methods may have surfaces enriched in the additive.

Downstream and upstream equipment utilised in the extrusion compounding and forming processes can include drying systems, gravimetric dosing and feeding systems, vacuum calibration/cooling water bath, haul-off systems and in-line measurement systems.

The articles herein can be manufactured by moulding of thermoplastic material (e.g., polymer/additive composition) in a mould and/or extruding the thermoplastic material through a customised extrusion die. The amount of shear the polymer melt experiences is controlled during either process to ensure a maximum concentration of additives resides at the resultant solid polymer surfaces.

### Boltorns—Hyperbranched Polyesters

Boltorn is a family of polyester hyperbranched materials that are generated through pseudo one-pot polycondensations of AB₂ monomer named 2,2-bismethylol propionic acid (bis-MPA) and from a multifunctional core, typically tetra-functional. The obtained hyperbranched polymer comprises a hydrophobic interior and hydrophilic hydroxyl functional outer layer. These commercially available materials are trademarked, with species including H20 through to H40, with structures as represented by FIG. 1 (b), depending on the generation/degree of branching. In contrast to Hybrane with multiple ester/amide bonds, the Boltorn species consist of a great number of interior esters and large number of peripheral hydroxyl groups, independent of pseudo generation. The Boltorn skeleton does not garner the necessary hydrophilicity required to solubilize in water nor are they sufficiently hydrophobic to dissolve in hydrophobic solvents (e.g.) ethyl acetate, ether etc. Therefore, to alter the properties of Boltorn materials, a more defined dendritic core-shell skeleton can be achieved by postfunctionalisation, e.g., chemical modification, with appropriate substituents. Such postfunctionalisation may result in commercially available products such as lipophilic U3000 (see FIG. 5(A)) with unsaturated fatty acid. These qualities alter the polar nature of the specific Boltorn species to be more soluble in less polar solvents. However, the overall polarity obtained with such a hydrophobic core, coupled with the currently available substituents of (a) fatty acids and/or (b) PEGs, do not alter the final characteristics of the polymer enough to yield the necessary combination of (a) heterogeneous structural configuration, (b) incompatibility with the host matrix and (c) amphiphilic, incompatible nature necessary to deliver a compact core-shell nanoparticle to the surface of a thermoformed article with the necessary surface characteristics.

### Hybranes—Polyesteramides

Hybrane is a family of polyesteramide hyperbranched materials that is grown through a polycondensation reaction between diisopropanelamine (DTPA) and a selected cyclic anhydride. The final property of the typically hydroxyl functional Hybrane is directly correlated to the careful selection of the anhydride monomer. This ability to tune the properties of Hybrane based HBP is demonstrated by considering the water solubility of the following three examples (1) S1200, (2) D2800 and (3) DEO7508500 (see FIG. 5(B)-FIG. 5(D)). (1) With Hybrane S1200, the choice of succinic anhydride as a component confers its water solubility as a consequence of (a) the peripheral hydroxyl groups combined with (b) the aliphatic amide bonds available in the interior. (2) Conversely, by choosing dodecenyl succinic anhydride the obtained Hybrane (D2800) is water insoluble even though peripheral hydroxyl groups are present. This is due to the overwhelming hydrophobicity that comes from the dodecenyl groups with “shield” the aliphatic amide bonds from interacting with water through secondary forces, thus preventing water solubility. (3) However, the replacement of the hydroxyl groups in D2800 with short polyethylene glycol groups produces an amphiphilic polymer, as present in Hybrane (DEO7508000), which is also water soluble. The DEO7508000 represents a core-shell structure which enables it to adapt its conformation depending on its surrounding.

### EXAMPLES

The following examples are intended to illustrate various aspects of the present disclosure without, however, being limiting, in nature. It is understood that the present disclosure encompasses additional embodiments and features consistent with the foregoing description and following examples.

### Example 1 Extruded Tubes

Six percent Boltorn H40 (a hyperbranched polyester) and six percent Hybrane PS2550 (hyperbranched polyester amine) were separately compounded with a Marlex 5502 HDPE as the host matrix in a 100K clean room using a Leistritz twin screw extruder—a ZSE 27 MAXX—40 L/D—a 27 mm diameter, 40 L/D twin screw compounder fed by up to 4 K-Tron gravimetric dosing unit and downstream with a 4-hole strand die, feeding a Rieter pelletising unit. The standard medium shear screw configuration was used with no melt filtration as standard. The permissible tolerance for each feeder during production was ±1.0% of the addition rate. Subsequently, each polymer blend was extruded to the dimensions listed in Table 1.

TABLE 1
Dimensional specifications applied during the extrusion process.

| Dimensions | in | mm |
|---|---|---|
| Inner diameter (ID) | 0.091 ± 0.002 | 2.31 ± 0.051 |
| Outer diameter (OD) | 0.130 ± 0.0025 | 3.30 ± 0.063 |
| Wall Thickness (REF) | 0.0195 ± 0.00125 | 0.495 ± 0.032 |
| Length | 59.055 ± 1.969 | 1500 |

Tubular samples of each extrusion (the control HDPE tube, and Tubes A and B containing the commercially available HBPs) were analysed with XPS³ (X-ray Photoelectron Spectroscopy, using a Kratos AXIS-165, Mono Al X-rays, referencing the NIST-XPS database, version 3.5), a technique which measures the chemical composition within a surface depth of nanometers³. FIG. 6(a) compares the spectra of (X), a high density polyethylene (HDPE) control tube against (Y), Tube A containing a 5 wt. % of a fourth generation hyperbranched polyester, Boltorn H40 in a HDPE host matrix and (Z), Tube B containing a 5 wt. % of a hyperbranched polyester amide, Hybrane PS2550 in a HDPE host matrix. The absence of a peak with a binding energy between 288-290 eV in the HDPE control tube when compared to the other two tubes confirmed the presence of hyperbranched polymers at the surface of both. Further analysis of Tube B, shown in FIG. 6(b), confirmed the presence of nitrogen (N) at both the inner and outer surfaces of the extruded tube, indicating that the hyperbranched polyester amide, Hybrane PS2550, resided at both surfaces and not in the bulk of the extrusion. ³ XPS spectra are, for the most part, quantified in terms of peak intensities and peak positions. The peak intensities measure how much of a material is at the surface, while the peak positions indicate the elemental and chemical composition. the best way to compare XPS intensities is via, so called, percentage atomic concentrations. The key feature of these percentage atomic concentrations is the representation of the intensities as a percentage, that is, the ratio of the intensity to the total intensity of electrons in the measurement.

This study was performed to demonstrate the ability of branched polymers to act as a vehicle in which to transport selected functional groups of the branched polymers to both the inner and outer surfaces of an extrusion.

A batch of each extruded tubing including the HDPE control and each extrusion comprising a hyperbranched polymer (Tubes A and B), were overmoulded with an ISO594 compatible luer. These overmoulded extrusions were then tested in accordance with ISO10555-1. The force at break was used to determine the Ultimate Tensile Strengths (UTS) for each material, measuring both the bond between the extrusion and the overmoulded Luer as well as the UTS of the raw extrusions, as exhibited in FIG. 8, wherein the left-hand portion of FIG. 8 shows the UTS values for the overmoulded extrusions, and the right-hand portion of FIG. 8 shows the UTS values for the raw extrusions. Analysis confirmed that there was a significant difference in UTS's obtained for the overmould bond compared to that of the raw extrusion for both the control and Extrusion B. The UTS of the bond between the luer and extrusion for the control was higher than that measured for the raw extrusion alone. In contrast, the UTS of the raw extrusion for Tube A showed no significant difference when compared to the overmoulded bond with the luer. Secondly, there was a significant difference in the UTS of both Tube A and Tube B when compared to the control tube, suggesting a slight decrease in UTS compared to the control. Thirdly, there was a significant difference in UTS for Tube A compared to Tube B, with Tube A having lower force at break compared to Tube B. Finally there was no significant difference in UTS of the overmould bond with Tube A compared with that of Tube B.

In summary, the addition of the hyperbranched polyester to HDPE matrix (Tube A) appeared to have more influence on the physical properties of the extrusion when compared to the addition of the hyperbranched polyester amide to HDPE matrix (Tube B). The overmoulded bond of Tube B and its luer behaved the same as the control, with UTS of the raw extruded tube increasing compared to that of the control. The Force at Break and UTS recorded were all well above the required ISO values (in this case for a IOF extrusion there is minimum requirement of at least 5 N force to break according to ISO10555-1). Therefore, this example indicates that the addition of HBP to the extrusion does not detrimentally effect the bulk mechanical properties of the tube or its ability to form a strong overmould bond, with results again well within the requirements of ISO standards.

### Example 2 DNT750PE Compression Moulded Sheets

Hybrane DEO7508500 (FIG. 5(D)) in a low weight percent (6 wt. %) was compounded with HDPE using a Leistritz twin screw extruder—a ZSE 27 MARX—40 L/D—a 27 mm diameter, 40 L/D twin screw compounder fed by up to 4 K-Tron gravimetric dosing unit and downstream with a 4-hole strand die, feeding a Rieter pelletising unit. The standard medium shear screw configuration was used with no melt filtration as standard and run according to data sheet parameters for the medical grade High Density Polyethylene (HDPE) polymer matrix into rods which were pelletized. The resultant pellets were analysed via Differential Scanning calorimetry (DSC), the curves of which revealed a single Tₘ for the resulting polymer (DNT750PE), suggesting miscibility between the two materials, as demonstrated in FIG. 9(b). The difference in enthalpy between the HDPE (184.8 Jg⁻¹) and DNT750PE (198.7 Jg⁻¹) is also presented, indicating an increase in the internal energy available in the system. These pellets were subsequently compression moulded between two heated platens (ca. 150° C.) under pressure (ca. 1000 psi) to produce thin sheets. The sheets were cut into the appropriate sizes according to the standard ASTM D1894 and tested.

Compressed sheets were also made from the raw polymer HDPE matrix resin, which acted as the control. At least 5 compressed samples for each material, as per the standard, were tested. The static (μₛ) and dynamic co-efficient of friction (μ_{d}) for each were recorded and average and standard deviations values calculated. The average μ_{d} obtained for the HDPE control and DNT750PE are compared against Teflon in FIG. 10. The results indicate the DEO750 additive has migrated to the surface of the thermoformed article and the PEG groups have contributed to a reduced μ_{d} for the compressed sheets of DNT750PE when compared to the HDPE control.

As an additive, the DEO7508500 is expected to alter its amphiphilic core-shell conformation as it migrates through a bulk polymer matrix, all the while adapting and changing its branched and compact structure. Without intending to be bound by theory, this is believed to be the mechanism at work in the DNT750PE thermoformed article presented in Example 2, whereby the Hybrane molecules migrate though the HDPE matrix by reversed core-shell mechanisms and upon reaching the surface, the dodecenyl component of the dendritic molecule intertwines in the HDPE matrix while the PEG component is exposed to the surface. It is this amphiphilic nature of the molecule, whereby the hydrophilic and hydrophobic elements work against each other, promoting (1) migration of the main molecule to the air/polymer interface with PEG exposed to the air and the dodecenyl constituent trapped in the HDPE matrix and (2) manipulation of the surface properties of the final thermoformed article.

### Example 3 Compounding & Film Extruding

The materials used in Example 2, a HDPE control matrix and a hyperbranched polyester amine (Hybrane DEO7508500) compounded using the same weight percent (6 wt. %) in a HDPE matrix, were compounded using a Leistritz twin screw extruder—a ZSE 27 MAXX—40 L/D—a 27 mm diameter, 40 L/D twin screw compounder fed by up to a K-Tron gravimetric dosing unit and downstream with a coat hanger split sheet die and a three roll mill with PTFE sheet fitted to all cylinders. The standard medium shear screw configuration was used with no melt filtration as standard whereby the screw speed (rpm) and feed rate/throughput (Kg/Hr) were varied. Whilst resultant properties varied with extrusion parameters, most specifically shear rate, acceptable parameters according to Table 2 resulted in extrusions with desired properties. The barrel temperature profile was held constant across the runs between 110-200° C. at intervals of 10° C. from zone to zone within the screw chamber. Extruded sheets with thicknesses greater than the walls of an extruded tube were subsequently made for each batch. These were then used to measure and interpret the effect of the process parameters on the coefficient of friction of the extruded article.

TABLE 2
Parameters.

| Batch | | Feedrate | Screw Speed |
|---|---|---|---|
| No. | Sample | Kg/Hr | rpm |
| RUN 3 | HDPE | 1.417 | 200 |
| | DNT750 | 0.085 | |
| RUN 3 Control | HDPE | 1.417 | |

Using ASTM D1894, the dynamic (μ_{d}) co-efficient of friction of each of these sheets was measured and compared to those obtained for the compression moulded sheets of the same material. The μ_{d} of the PTFE extruded sheets was again measured to ensure consistency. As can be seen in FIG. 10, there is a decrease in μ_{d} observed for each of the HDPE materials when results for the compressed sheets are compared to the extruded sheets. As was exhibited for the compressed sheets, the presence of the DEO7508500 additive in the HDPE matrix reduced the μ_{d} when compared to the raw HDPE matrix. The same batch of PTFE extruded sheet was used during each test set-up, with two batches of PTFE tested to ensure any variability between tests were eliminated. These results demonstrate that post-processing the DNT750PE material through thermoforming processes results in greater reductions in its μ_{d}, producing a value below that of PTFE (e.g., Teflon).

### Example 4 Extrusion of HDPE Tubes

Following the identification of optimal composition and process parameters in the production of HDPE modified sheet, work commenced on producing suitable tubing. Prior to mixing the DEO7508500 material into the HDPE virgin pellets, the latter were mechanically ground down to a flake of ˜1200 μm in order to increase the surface area of the material. This material was then mixed with the appropriate quantities of DEO7508500 and compounded in twin screw Liestritz compounding equipment to form strands which were subsequently pelletised, as described in Example 3.

Tubes with the modified compounded HDPE pellets using a 16 mm co-rotating Dr Collin GmbH Twin Screw 28:1 LID ratio with a K-Tron dosing system. Downstream equipment included a Dr. Collin tube die, Dr. Collin vacuum calibration tank with slotted vacuum bushing, Dr. Collin haul-off and a Zumback OD measurement system run according to the parameters listed in Table 3.

TABLE 3
Process parameters used to extrude HDPE tube with a Twin Screw extruder.

| | | | | | | Feed | | Ext Head | | | | | | | | |
|---|---|---|---|---|---|---|---|---|---|---|---|---|---|---|---|---|
| | | | | | | Arm | | Die | | Water | | Screw | | | Haul | Feed |
| | Z1 | Z2 | Z3 | Z4 | Z5 | 1 | 2 | 1 | 2 | bath | Pressure | Speed | Feed | Vac | off | Zone |
| Run | ° C. | | | | | ° C. | | | | ° C. | % | rpm | Kg/Hr | KPa | m/min | ° C. |
| Virgin | 220 | 235 | 245 | 250 | 250 | 250 | 250 | 260 | 255 | 30 | 235 | 50 | 3 | 39 | 2.5 | 30 |
| HDPE | | | | | | | | | | | | | | | | |
| Mod. | 220 | 235 | 245 | 250 | 250 | 250 | 250 | 260 | 255 | 30 | 215 | 50 | 3 | 39 | 2.25 | 30 |
| HDPE | | | | | | | | | | | | | | | | |

Virgin HDPE tubes were produced according to the parameters as presented in Table 3 with the twin screw extruder. Modified HDPE pellets were then fed into the co-rotating Dr Collin twin screw extruder according to the parameters in Table 3. Modified tubes were produced, with samples of both virgin and modified tubes.

Development of an additive, generically called Factor DNT, commenced. Numerous different linear additives were linked via different classes and types of branched polymer elements. In relation to the non-limiting example of reduced friction, the following gives examples of Factor DNT which resulted in co-efficient of friction similar to that of PTFE (e.g., Teflon).

### Branched Hybrid Polymers

Exploiting polar heterogeneity, branched hybrid polymers with components having specific functionally, can be constructed in order to be compatible enough with the host polymer to prevent phase separation and incompatible enough to enable migration of the branched hybrid polymer to the surface of the final thermoformed article. Migration may be facilitated through charge disparity with the host polymer and a compact structural configuration of the nanoscopic branched polymer element composite, whilst portions of the structure are compatible enough to ensure entrapment of the composite when it reaches the final surface. Once at the surface, the peripheral ends may provide the final article with the necessary surface properties.

### Example 5 Branched Hybrid DNT022PX (6 k-3G) Compression Moulded

To this end, as a non-limiting example, a branched hybrid polymer DNT022 was synthesized to include a linear hydrophilic PEG core linked to many peripheral hydrophilic PEG oligomers via branched polymer components in the form of a hyperbranched Boltorn G3 hydrophobic polyester species as shown in FIGS. 11(a) and 11(b).

Carboxylic acid functionalized methoxyl polyethylene glycol acid (O_{A}=m-PEG750COOH) (13.3 g) was dissolved in dicholoromethane (DCM) (100 ml) with a magnet stirrer in a round bottom flask. To activate the acid, N,N′-carbonyl diimidazole (CDI) was slowly added to the solution to excess, with the outlet for CO₂ by-product (molar ratio OH:CDI activated COOH [1:1.5]). The reaction was proceeded for 2 hours and monitored via ¹H-NMR to confirm full activation. Thereafter, a polyester HBP of G3 (5 g) was added to the reaction vessel and conducted for 15 hours. The completion of the reaction was confirmed by ¹H-NMR and ¹³C-NMR.

The reaction was quenched with deionized water, the solution of which was diluted with 400 ml of DCM. Subsequently, the reaction was washed with 4×50 ml of NaHCO₃ (aq. 10 wt. %) and dried with magnesium sulfate. The crude product was precipitated from DCM to ether and collected as white powder. ¹H-NMR, ¹³C-NMR and SEC were utilized to confirm the purity of DNT022.

The organic compound, 1,1′-Carbonyldiimidazole (CDI) (C₃H₃N₂)₂CO, which, in general is used as a coupling reagent to activate carboxylated molecules prior to their reaction with nucleophiles such as alcohols and amines, was employed in the reaction between the hydroxylated periphery of the hyperbranched polymers with PEG-COOH oligomers. The obtained hyperbranched polymers may be described as being functionalised with PEG oligomers. The PEG chains were enlisted to provide a hydrophilic surface on the final thermoformed surface. The absence of mPEG750-COOH and hydroxyl peaks (R—CH₂—OH) in the NMR spectra of the resultant material, as demonstrated in FIG. 12(a), suggests successful linkage of the linear PEG core with the methoxyl terminated PEG750 chains via the hyperbranched polyester linker. The molecular weight (M_{w}) of the resultant DNT022 was compared to that of the precursor as demonstrated in FIG. 12(b), showing a shift to a broader and higher M_{w}, confirming terminal pegylation of the hybrid.

Six weight percent (6 wt. %) DNT022 was compounded with PEBAX 7233 SA01 MED, moulded to produce rods of DNT022PX and subsequently pelletized. PEBAX control pellets also underwent the same compounding process. These DNT022PX and PEBAX control pellets were compression moulded between two heated compressed plates (ca. 200° C./1000 psi) to produce thin sheets made of DNT022PX and PEBAX, with the same process steps employed for each material. The surfaces of both articles were then analysed by Raman spectroscopy, which indicated that the surface of the DNT022PX article had over 20% more ether (—C—O—C—) groups (a constituent of PEG) at its surface compared to that of the control following immersion in deionised water, as shown in FIG. 13 (a). Therefore, the articles were immersed in deionised water prior to measurement of dynamic coefficient of friction (μ_{d}) according to ASTM D1894-11. As can be seen from FIG. 13 (b), μ_{d} for the DNT022PX was lower than the PEBAX control and PTFE, tested in the same conditions. Therefore, these results indicate that the make-up of the linear dendritic hyperbranched polymer, DNT022, enabled it to migrate through the PEBAX host matrix and position itself in the surface layers of the resultant thermoformed article, delivering the hydrophilic peripheral groups to the outer most surface of the final article.

### Example 6 Branched Hybrid (Various Generation Numbers & Chain Lengths) Extruded in PEBAX

Further to Example 4, as a non-limiting example, a matrix of materials, as shown in Table 4, similar to DNT022 (6kG3) were synthesized. These materials were made up of linear hydrophilic PEG cores varying in size to include 6 k, 10 k and 20 k, which were linked to many peripheral hydrophilic PEG oligomers via branched polymer components in the form of a hyperbranched polymers with either generation G3 or G5 hydrophobic polyester species.

TABLE 4
Matrix of Branched Hybrids.

| | Generation | Core Linear | Core | PEBAX |
|---|---|---|---|---|
| Code | Number | Chain Size | Functionality | Sheets |
| 6kG3 | 3 | 6 | 2 | 6kG3PX |
| 10kG3 | 3 | 10 | 2 | 10kG3PX |
| 20kG3 | 3 | 20 | 2 | 20kG3PX |
| 6kG5 | 5 | 6 | 2 | 6kG5PX |
| 10kG5 | 5 | 10 | 2 | 10kG5PX |
| 20kG5 | 5 | 20 | 2 | 20kG5PX |

FIG. 16(a)-16(c) include the number average molecular weight (M̅ₙ̅, the weight average molecular weight (M̅_̅{̅w̅}̅) and the dispersity (Ð) for each material. Monodisperse materials have a Ð value of 1, whilst values between 1.1-2.0 would be considered moderately polydisperse. From the graphs presented 6kG5 and Boltorn are similarly disperse, with 6kG5 (M̅_̅{̅w̅}̅ theoretically 67,700 g/mol) having a higher M̅_̅{̅w̅}̅ compared to the Bottom (theoretically 15,200 g/mol). The 10kG3 has the narrowest dispersity of these three additives but it again has a higher M̅_̅{̅w̅}̅ (theoretically 25,200 g/mol) than Boltorn. The information presented in FIG. 16 (a)-(c), together with FIGS. 15 (a) and (b) would suggest that migration through a host matrix to the surface would be easiest for the Boltorn additive.

Three point five weight percent (3.5 wt. %) and ten weight percent (10 wt. %) of each branched hybrid material in Table 4 was compounded and extruded with PEBAX 7233 SA01 MED, producing PEBAX sheets for each materials. PEBAX control sheets were produced using the same process steps. Samples of each sheet type article were immersed in water at 37° C. prior to measurement of μₛ and μ_{d} according to ASTM D1894-11. As can be seen from FIGS. 17 (a), (b) and (c), the results obtained for both μₛ and μ_{d} demonstrate the influence of generation number (Gₙ) and core chain length of the additive on the coefficient of friction of the extruded sheets. The effect of an increase in terminal end groups with an increase in generation number from G3 to G5, the theoretical structure of which is demonstrated in FIG. 15(d) (XkG5 to XkG3), appears to result in a slight decrease in frictional properties of the extrudate when compared against each other and the PEBAX control in FIG. 17(a), suggesting that the higher number of terminal end groups present in the G5 additive are influencing the effect on surface properties of the extrudate.

Conversely, when the length of the linear core chain is increased in each of these G3 and G5 additives, the results of which are presented in FIG. 17(b), it would appear that the additive with lower generation hyperbranched elements (10kG3) migrate more readily to the surface of the extrudate compared to the higher generation based additive (10kG5). Comparing the Boltorn H₂O P and 10kG3 results, the lower μ_{d} value obtained with the L-D HB additive would suggest increasing the distance between the hyperbranched elements results in a conformational structure which aids migration through the host matrix to the extrudate surface. A reduction in μ_{d} was also observed when 10kG5 and 10kG3 results are compared, with a lower μ_{d} obtained with 10kG3, the additive with a lower Gₙ, a trend opposite to that observed for the core linear chains of 6 k, in FIG. 17a. Therefore, in order to encourage migration of the additive to the surface and utilisation of terminal end groups when the additive reaches the extrudate surface, it would appear that additives suitable for various applications may strike a balance between:
additional terminal oligomers obtained from an increase in generation number (Gₙ),
restrictions imposed by such an increase in Gₙ on the migration of the additive to the surface of the host polymer matrix,
increasing the separation distance between the hyperbranched elements by varying the core linear chain length,
potential entrapment of additive in the host polymer matrix due to a lengthening core linear chain interacting with host polymer matrix chains.

In summary, results from FIGS. 17(a) and 17(b) coupled with a comparison of the structural conformation of each of the additives, as presented in FIGS. 15 (a), 15(b), 15(c) and 15(d) would suggest the structure of the additive has a considerable influence over migration of the additive through the host polymer matrix.

This observation is reinforced when considering the results as presented in FIG. 17(c). A slight decrease in μₛ and μ_{d} was observed with the addition of Boltorn H20P when compared to the PEBAX control. However larger drops in μₛ and μ_{d} were observed with linear dendritic hyperbranched (L-D HB) additives, the drop in which was more pronounced with increases in the length of the linear core of the L-D HB additive. The radial shaped Boltorn material would be expected to migrate to the surface of the extruded part more readily than the linear dendritic hyperbranched additives due to its compact structure and size. However comparing the results obtained with Boltorn H20P to those with 6kG3 and 10kG3, which have a comparably less compact conformational structure, lower friction properties are observed with both LD-HB additives. Additionally, an increase in the distance between the hyperbranched cores due to a longer core linear chain, 6kG3 verses 10kG3, would be expected to result in a reduction in segmental crowding around the hyperbranched. cores, enabling greater migration of terminal chains to the surface of the extrudate, with a resultant lower friction result. This ability to modify and control the migration of the additive to the surface via changes to its molecular structure provides the possibility to tune surface properties of the final extrudate. These reductions in friction properties are linked to conformational structures and decreases in segmental crowding of the hyperbranched elements of the additive.

It can be seen in FIG. 18(a) that G5 based additives the μₛ increases with increased core linear chain length from 6 k to 10 k and appears to reach a plateau comparing 10 k to 20 k results. A similar trend is observed for μ_{d} with both G3 and G5 based additives, as presented in FIG. 18(b), with a reduction in friction observed for a core chain length of 10 k when compared to the shorter 6 k and longer 20 k core chain lengths. In fact both the 10 k and 20 k G5 extrudates demonstrate μₛ values above that for the virgin PEBAX control. For G3 based additives the μₛ decreases compared to the virgin control with core chain lengths of 6 k and 10 k, however increase above the virgin value with a core chain length of 20 k. As discussed previously, separation of the hyperbranched elements may prevent these groups from acting on each other, thereby facilitating their superior migration through the host matrix. However the migration benefits due to such decreases in segmental crowding as a result of increases in core chain length, may be offset by entrapment of the core chain by/with chains in the host matrix when the core chain length increases above a certain length.

Elution testing performed on PEBAX and HDPE modified extruded sheets using human dermo fibroblasts, presented in FIG. 19 (a) and mouse macrophages, shown in FIG. 19 (b), demonstrate >85% cell viability after 72 hours for all the samples, clearly demonstrating extrudate of modified host matrices are non-toxic.

Whilst not being bound by theory a number of factors may influence the migration of the additives through a host polymer matrix in order to allow the additive to achieve a concentration gradient such that the additive is positioned predominantly in the surface layers of the resultant thermoformed article, delivering the peripheral groups of the additive to the outer most surface(s) of the final formed article. These factors may include, but are not limited to:
energy differences between the additive and the host polymer, e.g., producing segregation via enthalpic differences;
entropic differences between the additive and the host polymer via shearing imparted during the blending and thermoforming processes;
molecular size/weight of the additive;
structural conformation of the additive; and/or
lack of entanglements in/with the hyperbranched structure of the additive as compared to the structure of the host polymer.

### Example 7 HBP—OH Stabilised Silver Colloidal Particles

The following prophetic example illustrates the use of a linear dendritic hyperbranched polymer with peripheral hydroxyl groups to stabilise colloidal silver particles, which may have the ability to release microbicidal catonic silver species. In this example the following are used: polyester HBP of G3 with peripheral hydroxyl groups, silver nitrate (AgNO₃) as the active antibacterial agent, water as a solvent, and sodium borohydride as a reducing agent. Those skilled in the art of producing elemental nano- and micro-particles of noble metals will understand that a number of common reducing agents and silver salts would be suitable to form elemental silver particles in this way, and thus this example serves only to aid understanding of the process.
Step 1. Dissolve the polyester HBP of G3 with peripheral hydroxyl groups in distilled water in a round bottom flask with magnetic stirring, at a concentration of 0.3 mM.
Step 2. Add silver nitrate (AgNO₃) in a ratio of silver:HBP of 1:1 and stir the solution for 24 hours at room temperature, so as to ensure complete formation of the HBP-silver complex.
Step 3. Addition of sodium borohydride (NaBH₃) reducing agent generates colloidal silver particles, in a molar ratio of silver nitrate:sodium borohydride of 1:10, with vigorous stirring.
Step 4. Stir the reaction mixture for a further 1-4 hours, observing the formation of colloidal silver particles by a colour change in the solution to yellow, pink, red or brown.
Step 5. Dialyse the solution against water in a 2 kDa molecular weight cut off regenerated cellulose membrane to remove impurities, and isolate the resulting solution by freeze drying.
Step 6. Characterize the resulting solid by ¹H-NMR, FTIR and UV/vis spectroscopy.

### Example 8 Tube Extrusion of HBP—OH Stabilized Silver Colloidal Particles in a PEBAX Host

The following prophetic example illustrates preparation of a blend of linear dendritic hyperbranched polymer synthesized in Example 7 and PEBAX 7233 SA01 MED and preparation of antimicrobial tubing from the blend.
Step 1. Weigh out appropriate quantities of both the synthesized HBP—OH stabilized silver colloidal particles additive of Example 7 and PEBAX 7233 SA01 MED pellets to produce a 3.5 wt. % mixture.
Step 2. Mix the quantities of silver LD MB polymer and PEBAX 7233 SA01 MED pellets with a mechanical mixer.
Step 3. Extrude the silver LD HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 9 Synthesis of COOH-Functional HBP

The following prophetic example illustrates the synthesis of a linear dendritic hyperbranched polymer with peripheral carboxylic acid groups. In this example the following are used: polyester HBP of G3 and succinic anhydride as reactants, 4-dimethylaminopyridine (DMAP) as activator, dichloromethane (DCM), pyridine, water and diethyl ether as solvents, and sodium hydrogen sulfate (NaHSO₄) as a washing solution.
Step 1. Dissolve 50 g of the polyester HBP G3 in 100 mL dichloromethane (DCM) with 10 mL pyridine in a round bottom flask with magnetic stirring.
Step 2. Add 2.5 g 4-dimethylaminopyridine, followed by slow addition of 15.8 g succinic anhydride. Allow the reaction to proceed for 24 hours and confirm by ¹H- and ¹³C-NMR.
Step 3. Subsequently quench the reaction mixture with 200 mL deionised water and stir until full quenching could be observed by ¹³C-NMR
Step 4. Wash the reaction mixture with 3×100 mL NaHSO₄ and dry over magnesium sulfate. Isolate the COOH-functional HBP as a white powder by precipitation into diethyl ether. Further purify the solid by freeze drying.
Step 5. Characterise the product using ¹H-NMR and ¹³C-NMR.

### Example 10 Silver Complex with COOH—HBP

The following prophetic example illustrates the use of the HBP described in Example 9 to form complexes with silver cations. In this example the following are used: polyester HBP of G3 with carboxylic acid peripheral groups, silver nitrate (AgNO₃) as the active antibacterial agent and water as a solvent. Those skilled in the art of coordination chemistry will understand that a number of silver salts would be suitable to form elemental silver particles in this way, and thus this example serves only to aid understanding of the process.
Step 1. Dissolve 1 g of the polyester HBP G3 with COOH peripheral groups in 50 mL methanol in a round bottom flask with magnetic stirring.
Step 2. After complete dissolution, add an aqueous solution of silver nitrate with vigorous stirring, so that the molar ratio of HBP:silver nitrate was 1:4.
Step 3. After 2 hours stirring, remove the solvents in vacuo and further purify the material via freeze drying.

### Example 11 Tube Extrusion of Silver Complex with COOH—HBP in a PEBAX Host

The following prophetic example illustrates preparation of a blend of linear dendritic hyperbranched polymer synthesized in Example 10 and PEBAX 7233 SA01 MED and preparation of antimicrobial tubing from the blend.
Step 1. Weigh out appropriate quantities of both the synthesized silver complex with the COOH—HB polymer of Example 10 and PEBAX 7233 SA01 MED pellets to produce a 3.5 wt. % mixture.
Step 2. Mix the quantities of silver LD HB polymer and PEBAX 7233 SA01 MED pellets with a mechanical mixer.
Step 3. Extrude the silver HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 12 Synthesis of Amine Functional-HBP

The following prophetic example illustrates the synthesis of a linear dendritic hyperbranched polymer with peripheral amine groups. In this example the following are used: polyester HBP of G3 and boc (tert-butyloxycarbonyl) protected beta-alanine anhydride as reactants, 4-dimethylaminopyridine (DMAP) as activator, dichloromethane (DCM), pyridine, water and diethyl ether as solvents, and sodium hydrogen sulfate (NaHSO₄) as a washing solution.
Step 1. Dissolve 50 g of the polyester HBP G3 in 100 mL dichloromethane (DCM) with 10 mL pyridine in a round bottom flask with magnetic stirring.
Step 2. Add 2.5 g 4-dimethylaminopyridine, followed by slow addition of 60 g boc-protected beta-alanine anhydride. Allow the reaction to proceed for 24 hours and confirm by 1H- and 13C-NMR.
Step 3. Subsequently quench the reaction mixture with 200 mL deionised water and stir until full quenching is observed by ¹³C-NMR
Step 4. Wash the reaction mixture with 3×100 mL NaHSO₄ and dry over magnesium sulfate. Isolate the COOH-functional HBP as a white powder by precipitation into diethyl ether. Further purify the solid by freeze drying.
Step 5. Characterise the product using ¹H-NMR and ¹³C-NMR.

### Example 13 HBP—NH₂ Stabilized Silver Colloidal Particles

The following prophetic example illustrates the use of the HBP described in Example 12 to stabilise colloidal silver particles, which may have the ability to release microbiocidal cationic silver species. In this example the following are used: polyester HBP of G3 with peripheral amine groups, silver nitrate (AgNO₃) as the active antibacterial agent, water and methanol as solvents, and sodium borohydride as a reducing agent. Those skilled in the art of producing elemental nano- and micro-particles of noble metals will understand that a number of common reducing agents and silver salts would be suitable to form elemental silver particles in this way, and thus this example serves only to aid understanding.
Step 1. Dissolve the polyester HBP of G3 with peripheral amine groups, as described in Example 12, in distilled water in a round bottom flask, with magnetic stirring, at a concentration of 0.4 mM.
Step 2. Add silver nitrate (AgNO₃) solution in water (120 mM) such that the ratio of silver:HBP is 25:1, and stir the solution for 30 minutes.
Step 3. Through the addition of sodium borohydride (NaBH₃) reducing agent solution in water/methanol (v/v=1/2), in a molar ratio of silver nitrate:sodium borohydride of 1:1.5, colloidal silver particles were generated with vigorous stirring.
Step 4. Stir the reaction mixture for a further 2 hours, to complete the reaction.
Step 5. Dialyse the solution against water in a 2 kDa molecular weight cut off regenerated cellulose membrane to remove impurities, and isolate the resulting solution by freeze drying.
Step 6. Characterise the resulting solid by 1H-NMR, FTIR and UV/vis spectroscopy.

### Example 14 Tube Extrusion of HBP—NH₂ Stabilized Silver Colloidal Particles in a PEBAX Host

The following prophetic example illustrates preparation of a blend of the silver hyperbranched polymer synthesized in Example 13 HBP—NH₂ stabilized silver colloidal particles and PEBAX 7233 SA01 MED and preparation of antimicrobial tubing from the blend.
Step 1. Weight out appropriate quantities of both the synthesized HBP—NH₂ stabilized silver colloidal particles and PEBAX 7233 SA01 MED pellets to produce a 3.5 wt. % mixture.
Step 2. Mix the quantities of silver HB polymer and PEBAX 7233 SA01 MED pellets with a mechanical mixer.
Step 3. Extrude the silver HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 15 Amphiphilic PEG HBP with PEG750COOH and PFHA

The following prophetic example illustrates the synthesis of a linear dendritic hyperbranched polymer with both hydrophilic and hydrophobic terminal end groups. In this example the following are used: m-PEG750COOH, perfluoroheptanoic acid and polyester HBP of G3 with peripheral hydroxyl groups as reactants, dicholoromethane (DCM) as solvent and N,N′-carbonyl diimidazole (CDI) as activator.
Step 1. Dissolve 67.2 grams of carboxylic acid functionalized methoxyl polyethylene glycol acid (OA=m-PEG750COOH) and 28.8 grams perfluoroheptanoic acid (CF₃(CF₂)₆CO₂H) in dicholoromethane (DCM) (200 ml) with a magnet stirrer in a round bottom flask
Step 2. Slowly add 24 grams N,N′-carbonyl diimidazole (CDI) to the solution to excess activate the acid, with the outlet for CO₂ by-product (molar ratio OH:CDI activated COOH [1:1.5]).
Step 3. Allow the reaction to proceed for 2 hours, monitoring via ¹H-NMR to confirm full activation.
Step 4. On full activation, add 50 grains of polyester HBP of G3 to the reaction vessel and allow to proceed for 15 hours. Confirm completion of the reaction by ¹H-NMR and ¹³C-NMR.
Step 5. Quench the reaction with deionized water.
Step 6. Dilute this solution with 200 ml of DCM.
Step 7. Wash the reaction with 4×150 ml of NaHCO₃ (aq. 10 wt. %) and dry with magnesium sulfate.
Step 8. Precipitate the crude product from DCM to diethyl ether and collect.
Step 9. Confirm sample purity utilising ¹H-NMR, ¹³C-NMR and SEC.

### Example 16 Tube Extrusion of Amphiphilic PEG HBP with PEG750COOH and PFHA in a PEBAX Host

The following prophetic example illustrates preparation of a blend of the amphiphilic hyperbranched polymer synthesized in Example 15 Amphiphilic PEG HBP with PEG750COOH and PFHA Example 13 HBP—NH₂ stabilized silver colloidal particles and PEBAX 7233 SA01 MED and preparation of antimicrobial tubing from the blend.
Step 1. Weigh out appropriate quantities of both the synthesized amphiphilic HBP and PEBAX 7233 SA01 MED pellets to produce a 3.5 wt. % mixture.
Step 2. Mix the quantities of the amphiphilic HB polymer and PEBAX 7233 SA01 MED pellets with a mechanical mixer.
Step 3. Extrude the amphiphilic HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 17 Hydrophobically Modified PEG HBP with Stearic Acid

The following prophetic example illustrates the synthesis of a linear dendritic hyperbranched polymer with hydrophobic terminal end groups. In this example the following are used: stearic acid and polyester HBP of G3 with peripheral hydroxyl groups as reactants, dicholoromethane (DCM) as solvent and N,N′-carbonyl diimidazole (CDI) as activator.
Step 1. Dissolve 39.3 grams of stearic acid in dicholoromethane (DCM) (200 ml) with a magnet stirrer in a round bottom flask
Step 2. Slowly add 24 grams N,N″-carbonyl diimidazole (CDI) to the solution to excess activate the acid, with the outlet for CO₂ by-product (molar ratio OH:CDI activated COOH [1:1.5]).
Step 3. Allow the reaction to proceed for 2 hours, monitoring via ¹H-NMR to confirm full activation.
Step 4. On full activation, add 50 grams of polyester HBP of G3 to the reaction vessel and allow to proceed for 15 hours. Confirm completion of the reaction by ¹H-NMR and ¹³C-NMR.
Step 5. Quench the reaction with deionized water.
Step 6. Dilute this solution with 200 ml of DCM.
Step 7. Wash the reaction with 4×1 ml of NaHCO₃ (aq. 10 wt. %) and dry with magnesium sulfate.
Step 8. Precipitate the crude product from DCM to diethyl ether and collect.
Step 9. Confirm sample purity utilising ¹H-NMR, ¹³C-NMR and SEC.

### Example 18 Tube Extrusion of Hydrophobically Modified PEG HBP with Stearic Acid in a PEBAX Host

The following prophetic example illustrates preparation of a blend of the hydrophobic hyperbranched polymer synthesized in Example 17 Hydrophobically modified PEG HBP with stearic acid and PEBAX 7233 SA01 MED and preparation of antimicrobial tubing from the blend.
Step 1. Weight out appropriate quantities of both the synthesized hydrophobic HBP and PEBAX 7233 SA01 MED pellets to produce a 3.5 wt. % mixture.
Step 2. Mix the quantities of the hydrophobic HB polymer and PEBAX 7233 SA01 MED pellets with a mechanical mixer.
Step 3. Extrude the hydrophobic HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 19 Amphiphilic PEG HBP with PEG750COOH and Stearic Acid

The following prophetic example illustrates the synthesis of a linear dendritic hyperbranched polymer with both hydrophilic and hydrophobic terminal end groups. In this example the following are used: m-PEG750COOH, stearic acid and polyester HBP of G3 with peripheral hydroxyl groups as reactants, dicholoromethane (DCM) as solvent and N,N″-carbonyl diimidazole (CDI) as activator.
Step 1. Dissolve 67.2 grams of carboxylic acid functionalized methoxyl polyethylene glycol acid (OA=m-PEG750COOH) and 19.7 grams stearic acid in dicholoromethane (DCM) (200 ml) with a magnet stirrer in a round bottom flask
Step 2. Slowly add 24 grams N,N″-carbonyl diimidazole (CDI) to the solution to excess activate the acid, with the outlet for CO₂ by-product (molar ratio OH:CDI activated COOH [1:1.5]).
Step 3. Allow the reaction to proceed for 2 hours, monitoring via 1H-NMR to confirm full activation.
Step 4. On full activation, add 50 grams of polyester HBP of G3 to the reaction vessel and allow to proceed for 15 hours. Confirm completion of the reaction by 1H-NMR and 13C-NMR.
Step 5. Quench the reaction with deionized water.
Step 6. Dilute this solution with 200 ml of DCM.
Step 7. Wash the reaction with 4×150 ml of NaHCO₃ (aq. 10 wt. %) and dry with magnesium sulfate.
Step 8. Precipitate the crude product from DCM to diethyl ether and collect.
Step 9. Confirm sample purity utilising 1H-NMR, 13C-NMR and SEC.

### Example 20 Tube Extrusion of Amphiphilic PEG HBP with PEG750COOH and Stearic Acid in a PEBAX Host

The following prophetic example illustrates preparation of a blend of the amphiphilic hyperbranched polymer synthesized in Example 19 Amphiphilic PEG HBP with PEG750COOH and stearic acid and PEBAX 7233 SA01 MED and preparation of antimicrobial tubing from the blend.
Step 1. Weight out appropriate quantities of both the synthesized amphiphilic HBP and PEBAX 7233 SA01 MED pellets to produce a 3.5 wt. % mixture.
Step 2. Mix the quantities of the amphiphilic HB polymer and PEBAX 7233 SA01 MED pellets with a mechanical mixer.
Step 3. Extrude the amphiphilic HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

### Example 21 Hydrophobically Modified PEG HBP with PFHA

The following prophetic example illustrates the synthesis of a linear dendritic hyperbranched polymer with hydrophobic terminal end groups. In this example the following are used: perfluoroheptanoic acid and polyester HBP of G3 with peripheral hydroxyl groups as reactants, dicholoromethane (DCM) as solvent and N,N′-carbonyl diimidazole (CDI) as activator.
Step 1. Dissolve 57.6 grams of perfluoroheptanoic acid (CF₃(CF₂)₆CO₂H) in dicholoromethane (DCM) (200 ml) with a magnet stirrer in a round bottom flask
Step 2. Slowly add 24 grams N,N′-carbonyl diimidazole (CDI) to the solution to excess activate the acid, with the outlet for CO₂ by-product (molar ratio OH:CDI activated COOH [1:1.5]).
Step 3. Allow the reaction to proceed for 2 hours, monitoring via 1H-NMR to confirm full activation.
Step 4. On full activation, add 50 grams of polyester HBP of G3 to the reaction vessel and allow to proceed for 15 hours. Confirm completion of the reaction by ¹H-NMR and ¹³C-NMR.
Step 5. Quench the reaction with deionized water.
Step 6. Dilute this solution with 200 ml of DCM.
Step 7. Wash the reaction with 4×1 ml of NaHCO₃ (aq. 10 wt. %) and dry with magnesium sulfate.
Step 8. Precipitate the crude product from DCM to diethyl ether and collect.
Step 9. Confirm sample purity utilising ¹H-NMR, ¹³C-NMR and SEC.

### Example 22 Tube Extrusion of Hydrophobically Modified PEG HBP with PFHA in a PEBAX Host

The following prophetic example illustrates preparation of a blend of the hydrophobic hyperbranched polymer synthesized in Example 21 Hydrophobically modified PEG HBP with PFHA and PEBAX 7233 SA01 MED and preparation of antimicrobial tubing from the blend.
Step 1. Weigh out appropriate quantities of both the synthesized hydrophobic HBP and PEBAX 7233 SA01 MED pellets to produce a 3.5 wt. % mixture.
Step 2. Mix the quantities of the hydrophobic HB polymer and PEBAX 7233 SA01 MED pellets with a mechanical mixer.
Step 3. Extrude the hydrophobic HB polymer/PEBAX 7233 blend through a twin screw extruder with an appropriate die to produce a strand and subsequently pelletize the resultant strand.
Step 4. Feed the compounded blended pellets into an extruder fitted with an appropriate die according to the tube specifications required and at an appropriate temperature, avoiding degradation of both materials, producing tube with antimicrobial characteristics at its surface.

The invention is not limited to the embodiments hereinbefore described which may be varied in detail.

1. A method for producing a thermoformed article with enhanced properties, the method comprising the steps of:—
introducing an additive into a host polymer to form a polymer/additive composition; and
thermoforming the polymer/additive composition into the article so that the additive migrates to the surface of the thermoformed article,
the additive comprising
a polydispersed hyperbranched polymer (HBP) having at least two reactive groups, the polydispersed hyperbranched polymer being linked to a plurality of oligomer chains; or
a branched monodispersed dendritic polymer (DP) having at least two reactive groups, the branched monodispersed dendritic polymer being linked to a plurality of oligomer chains.

2. A method as claimed in claim 1 wherein the thermoforming is effected by extrusion forming.

3. A method as claimed in claim 2 wherein the extrusion forming comprises use of a single screw extruder or a twin screw extruder.

4. A method as claimed in claim 1 comprising forcing the molten mixture through a die.

5. A method as claimed in claim 1 comprising the step of heat treating the thermoformed article.

6. A method as claimed in claim 5 wherein the heat treatment is carried out at a temperature between room temperature and the glass transition temperature (Tg) of the host polymer.

7. A method as claimed in claim 1 comprising the step of blending the additive with the host polymer prior to thermoforming.

8. A method as claimed in claim 7 wherein blending is selected from the group comprising:—
mixing;
melt blending including extrusion compounding; and
solution blending comprising mixing said host polymer with said additive in a mutual solvent followed by dispersion blending.

9. A method as claimed in claim 1 wherein the additive comprises greater than 30 carbon atoms.

10. A method as claimed in claim 1 wherein the polydispersed hyperbranched polymer or the monodispersed dendritic polymer have short cores with two or more reactive groups and six or more reactive peripheral groups linked to a plurality of short oligomers, OA, where A represents a monomer.

11. A method as claimed in claim 1 wherein the polydispersed hyperbranched polymer or the monodispersed dendritic polymer have short cores with two or more reactive groups and six or more reactive peripheral groups linked to a number of short oligomers, OA, OC, where A and C each represent a monomer, present in a ratio OA:OC of from 1:100 to 100:1.

12. A method as claimed in claim 1 wherein the additive comprises a core linear chain.

13. A method as claimed in claim 12 wherein the core linear chain comprises at least one reactive group.

14-27. (canceled)

28. A method as claimed in claim 1 wherein the host polymer is a polymer selected from one or more of the group comprising polyolefins, polystyrenes, polyesters, polyamides polyethers, polysulfones, polycarbonates, polyureas, polyurethanes, polysiloxanes and thermoplastic polymers including blends of thermoplastic polymers with other thermoplastics or copolymers or blends thereof and thermoplastic elastomers including blends of thermoplastic polymers with other thermoplastics or copolymers or blends thereof.

29-40. (canceled)

41. An additive for a thermoformable polymer matrix comprising:—
a polydispersed hyperbranched polymer (HBP) having at least two reactive groups, the polydispersed hyperbranched polymer being linked to a plurality of oligomer chains; or
a branched monodispersed dendritic polymer (DP) having at least two reactive groups, the branched monodispersed dendritic polymer being linked to a plurality of oligomer chains.

42. An additive as claimed in claim 41 wherein the HBP or DP has greater than 30 carbon atoms.

43-80. (canceled)

81. A thermoformed article comprising a host polymer and an additive comprising:—
a polydispersed hyperbranched polymer (HBP) having at least two reactive groups, the polydispersed hyperbranched polymer being linked to a plurality of oligomer chains; or
a branched monodispersed dendritic polymer (DP) having at least two reactive groups, the branched monodispersed dendritic polymer being linked to a plurality of oligomer chains,
wherein a surface of the article is enriched in the additive with respect to a bulk of the article.

82-85. (canceled)

86. A thermoformed article as claimed in claim 81 wherein the article is a medical device.

87-88. (canceled)

89. A thermoformed article as claimed in claim 81 wherein a surface property selected from one or more of surface tension, surface energy, anti-microbial, anti-thrombogenic, anti-inflammatory, and radiopacity is modified compared to the surface property of the host polymer without the additive.

90. A thermoformed article as claimed in claim 81 wherein the host polymer is a polymer selected from one or more of the group comprising polyolefins, polystyrenes, polyesters, polyamides, polyethers, polysulfones, polycarbonates, polyureas, polyurethanes, polysiloxanes and thermoplastic polymers including blends of thermoplastic polymers with other thermoplastics or copolymers or blends thereof and thermoplastic elastomers including blends of thermoplastic polymers with other thermoplastics or copolymers or blends thereof.
